(19) United States
(12) Patent Application Publication
(10) Pub. No.: US 2022/0118123 A1
(43) Pub. Date: Apr. 21, 2022

(54) COMBINATION OF AR ANTAGONISTS AND TARGETED THORIUM CONJUGATES

(71) Applicants: Bayer Aktiengesellschaft, Leverkusen (DE); BAYER AS, Oslo (NO)

(72) Inventors: Stefanie HAMMER, Berlin (DE); Urs Beat HAGEMANN, Glienicke/Nordbahn (DE); Bernard HAENDLER, Berlin (DE); Pascale LEJEUNE, Toulouse (FR); Sabine ZITZMANN-KOLBE, Berlin (DE); Christoph SCHATZ, Berlin (DE); Jenny KARLSSON, Oslo (NO)

(73) Assignees: Bayer Aktiengesellschaft, Leverkusen (DE); BAYER AS, Oslo (NO)

(21) Appl. No.: 17/310,759

(22) PCT Filed: Feb. 17, 2020
(86) PCT No.: PCT/EP2020/054113

§ 371 Date: Aug. 20, 2021

(30) Foreign Application Priority Data
Feb. 22, 2019 (EP) 19158777.3

(51) Int. Cl. A61K 51/10; A61K 31/7088; A61K 31/277; A61K 31/4166; A61K 31/4439; A61K 31/58; A61K 31/4155; A61K 31/573; A61K 31/167; A61K 31/501; A61K 31/222; A61K 31/4192; A61K 31/4188; A61K 31/496; A61K 31/4164; A61P 35/00

(52) U.S. Cl. CPC A61K 51/1093; A61P 35/00; A61K 31/277; A61K 31/4166; A61K 31/4439; A61K 31/58; A61K 31/4155; A61K 31/573; A61K 31/167; A61K 31/501; A61K 31/222; A61K 31/4192; A61K 31/4188; A61K 31/496; A61K 31/4164; A61K 31/7088

(57) ABSTRACT

The present invention covers combinations of at least two components, component A and component B, comprising component A being PSMA-TTC, and component B being an antiandrogen selected form AR antagonists such as from cyproterone acetate, bicalutamide, flutamide, nilutamide, enzalutamide, apalutamide, darolutamide or keto-darolutamide, or an AR degrader such as ARV-110, or an ARN-terminal domain binder such as EPI-506, or an antisense oligonucleotide that reduces AR expression such as EZN-4176 or AZD-5312, or an androgen synthesis inhibitor such as abiraterone, particularly abiraterone acetate, seviteronel, galeterone, orteronel or ketoconazole, or a dual AR antagonist and androgen synthesis inhibitor such as ODM-204. Another aspect of the present invention covers the use of such combinations as described herein for the preparation of a medicament for the treatment or prophylaxis of a disease, particularly for the treatment of a hyper-proliferative disease.

The present invention relates to combinations of at least two components, component A and component B, component A being PSMA-TTC, and component B being an AR antagonist.

Another aspect of the present invention relates to the use of such combinations as described herein for the preparation of a medicament for the treatment or prophylaxis of a disease, particularly for the treatment of cancer.

Yet another aspect of the present invention relates to methods of treatment or prophylaxis of a cancer in a subject, comprising administering to said subject a therapeutically effective amount of a combination as described herein.

Further, the present invention relates to a kit comprising a combination of:
component A, as defined supra, or a solvate or hydrate thereof;
one or more components B, as defined herein, or a physiologically acceptable salt, solvate, hydrate or stereoisomer thereof; and, optionally
one or more pharmaceutical agents C;
   in which optionally either or both of said components A and B are in the form of a pharmaceutical formulation which is ready for use to be administered simultaneously, concurrently, separately or sequentially.

Component A preferably is administered by the intravenous route.

Component B may be administered by the oral, intravenous, topical, local installations, intraperitoneal or nasal route.

### BACKGROUND TO THE INVENTION

Cancer is the second most prevalent cause of death in the United States, causing 450,000 deaths per year. While substantial progress has been made in identifying some of the likely environmental and hereditary causes of cancer, there is a need for additional therapeutic modalities that target cancer and related diseases. In particular there is a need for therapeutic methods for treating diseases associated with dysregulated growth/proliferation.

Cancer is a complex disease arising after a selection process for cells with acquired functional capabilities like enhanced survival/resistance towards apoptosis and a limitless proliferative potential. Thus, it is preferred to develop drugs for cancer therapy addressing distinct features of established tumors.

Several alpha-emitters, such as Terbium-149 (149Tb), Astatine-211 (211At), Bismuth-212 (212Bi), Bismuth-213 (213Bi), Actinium-225 (225Ac), Radium-223 (223Ra), Radium-224 (224Ra), or Thorium-227 (227Th), have been investigated and/or commercialised for use as radiopharmaceuticals. In particular, the use of ‘tissue-targeting’ radiopharmaceuticals has meant that the radioactive nucleus can be delivered to the target cell (for example a cancerous cell) with an improved accuracy, thus minimising unwanted damage to surrounding tissue and hence minimising side effects. Tissue-targeting radiopharmaceuticals are typically conjugates in which the radiopharmaceutical moiety is linked to a targeting unit, for example via a chelator. The targeting unit (for example, an antibody) guides the radiopharmaceutical to the desired cell (by targeting a particular antigen on a cancer cell for example) such that the alpha radiation can be delivered in close proximity to the target. A small number of elements can be considered “self targeting” due to their inherent properties. Radium, for example, is a calcium analogue and targets bone surfaces by this inherent nature.

One particular class of tissue-targeting radiopharmaceuticals is Targeted Thorium Conjugates (TTCs), in which alpha-emitting thorium-227 (Th-227) nuclei are connected to tumor-targeting moieties such as antibodies. The radioactive pharmaceutical exploits the unique properties of elements that emit alpha particles, and the targeting properties of the conjugates help to minimise undesirable side effects.

Androgen receptor (AR) antagonists such as enzalutamide are effective in improving overall survival in castration-resistant prostate cancer (CRPC) patients. However, not all patients respond to these therapies, and even responders usuallydevelop resistance and experience disease progression after some time (Giacinti et al. Anticancer Res 2018 DOI: 10.21873/anticanres.12953). Multiple growth-promoting and survival pathways interact with AR signaling and are involved in prostate cancer (reviewed in Nevedomskaya et al. Int J Mol Sci 2018 DOI: 10.3390/ijms19051359). They include the PI3K/AKT/mTOR pathway as well as DNA repair pathways. In line with this, clinical benefit has been demonstrated for the combination of radiotherapy with androgen deprivation therapy in high-risk localized prostate cancer (Tosco et al. Eur Urol 2019 DOI: 10.1016/j.eururo.2018.07.027). Yet, failure of this combination may arise from insufficient blockade of androgen signaling. Thus new modalities to better inhibit androgen signaling and overcome resistance are still warranted (Crawford et al. 2018, J Urol DOI: 10.1016/j.juro.2018.04.083).

Beside AR antagonists which directly compete with the natural androgens testosterone and dihydrotestosterone for activation of the AR, compounds addressing other targets involved in androgen signaling have shown efficacy in prostate cancer models and in some cases in the clinic (Crawford et al. 2018, J Urol DOI: 10.1016/j.juro.2018.04.083; Nevedomskaya et al. Int J Mol Sci 2018 DOI: 10.3390/ijms19051359). They include compounds that bind to the AR N-terminal domain, antisense oligonucleotides that reduce AR expression and inhibitors of the androgen synthesis pathway, Recently, advances have been made in targeting metastatic prostate cancer using radiotherapy directed against prostate specific membrane antigen PSMA. Combining PSMA targeting agents with AR blockade is a potentially interesting approach, as AR blockade has been shown to increase levels of PSMA in prostate cancer models and in patients (Lueckerath et al. EJNMMI, 2018, Hope et al. JNM_2017 DOI: 10.2967/jnumed.116.181800). Thus, combination of PSMA-TTC with AR antagonists may be attractive due to a dual mode-of-action mechanism. First, increasing PSMA target levels by AR antagonist treatment may increase tumor targeting by PSMA-TTC. In addition, a weakening of the DNA damage response by AR antagonist treatment may increase intrinsic sensitivity of the tumors to PSMA-TTC. Yet, preclinical experiments combining the AR antagonist enzalutamide with PSMA-targeted radioligand therapy using the beta emitter 177Lu-PSMA-617 could so far not demonstrate synergistic anti-tumor effects in a C4-2 xenograft model (Lueckerath et al). Combinations of antibody-based targeted alpha therapy with AR antagonists has so far not been explored.

While considerable advances have been made over the last few years in the field of targeted radiopharmaceuticals, it would be of considerable advantage to provide further targeted therapeutic methods with increased efficiency. In particular, even with efficient targeting, there is a limit to the amount of radionuclide which can be administered to a subject without causing intolerable side-effects such as myelo-suppression. It would be of considerable benefit to provide a therapeutic method or a method of utilising such radionuclides which could enhance the efficacy of the medicament without requiring a higher dose of radiopharmaceutical.

The present inventors have now established that combinations of PSMA-TTC with AR antagonists can improve the therapeutic efficiency of PSMA-TTC in prostate cancer models. In particular, the combination treatment of the present invention may result in an additive, super-additive or synergistic interaction between PSMA-TTC and at least one AR antagonist, and may be employed to treat prostate cancer at various stages. A key advantage of the combination therapy of the present invention is the synergistic effect of the AR antagonist and PSMA-TTC targeting PSMA positive prostate cancer. The compounds work in tandem to increase the treatment effectiveness by targeting two different mechanisms, one being the induction of the PSMA level by the AR antagonist, thus increasing targeted delivery of the alpha emitter, the other one being the reduction in DNA repair mechanisms which will render PSMA-TTC treatment more efficacious.

The state of the art does not disclose the combinations of the present invention comprising a PSMA-TTC and an AR antagonist.

### SUMMARY OF THE INVENTION

Surprising effects in an in vivo tumor model were observed when administering PSMA-TTC in combination with an AR antagonist such as enzalutamide or darolutamide (ODM-201). The therapeutic efficacy of the combination described in the present invention has shown superiority to the efficacy achieved by the corresponding doses of AR antagonist or PSMA-TTC alone.

Therefore, in accordance with a first aspect, the present invention provides combinations of at least two components, component A and component B, comprising component A being PSMA-TTC described infra,

and component B being an AR antagonist, particularly an AR antagonist selected from cyproterone acetate, bicalutamide, flutamide, nilutamide, enzalutamide, apalutamide, darolutamide or keto-darolutamide, or an AR degrader such as ARV-110, or an AR N-terminal domain binder such as EPI-506, or an antisense oligonucleotide that reduces AR expression such as EZN-4176 or AZD-5312, or an androgen synthesis inhibitor such as abiraterone, particularly abiraterone acetate, seviteronel, galeterone, orteronel or ketoconazole, or a dual AR antagonist and CYP17A inhibitor such as ODM-204.

The combinations comprising at least two components A and B, particularly two components, as described herein, are also referred to as “combinations of the present invention”.

Further, the present invention covers a kit comprising:

component A: PSMA-TTC as described herein, a hydrate, a solvate thereof;
component B: one or more compounds that block AR function or inhibit androgen synthesis as described herein,
in which kit optionally either or both of said components A and B in any of the above-mentioned combinations are in the form of a pharmaceutical composition which is ready for use to be administered simultaneously, concurrently, separately or sequentially.

The components may be administered independently of one another by the oral, intravenous, topical, local installations, intraperitoneal or nasal route.

In accordance with another aspect, the present invention concerns the combinations as described herein for use in the treatment of for the treatment or prophylaxis of a disease, preferably a hyper-proliferative disease as described infra.

In accordance with another aspect, the present invention covers the use of such combinations as described herein for the preparation of a medicament for the treatment or prophylaxis of a disease, preferably a hyper-proliferative disease as described infra.

In accordance with another aspect, the present invention concerns methods for the treatment and/or prophylaxis of a disease, preferably a hyper-proliferative disease as described infra, using an effective amount of the combinations as described herein.

### DETAILED DESCRIPTION OF THE INVENTION

The terms as mentioned in the present text in context with compounds of general formula (I) or (Ib) have the following meanings:

The term “halogen atom”, “halo-” or “Hal-” is to be understood as meaning a fluorine, chlorine, bromine or iodine atom.

The term “C₁-C₆-alkyl” is to be understood as meaning a linear or branched, saturated, monovalent hydrocarbon group having 1, 2, 3, 4, 5, or 6 carbon atoms, e.g. a methyl, ethyl, propyl, butyl, pentyl, hexyl, iso-propyl, iso-butyl, sec-butyl, tert-butyl, iso-pentyl, 2-methylbutyl, 1-methylbutyl, 1-ethylpropyl, 1,2-dimethylpropyl, neo-pentyl, 1,1-dimethylpropyl, 4-methylpentyl, 3-methylpentyl, 2-methylpentyl, 1-methylpentyl, 2-ethylbutyl, 1-ethylbutyl, 3,3-dimethylbutyl, 2,2-dimethylbutyl, 1,1-dimethylbutyl, 2,3-dimethylbutyl, 1,3-dimethylbutyl, or 1,2-dimethylbutyl group, or an isomer thereof. Particularly, said group has 1, 2, 3 or 4 carbon atoms (“C₁-C₄-alkyl”), e.g. a methyl, ethyl, propyl, butyl, iso-propyl, iso-butyl, sec-butyl, tert-butyl group, more particularly 1, 2 or 3 carbon atoms (“C₁-C₃-alkyl”), e.g. a methyl, ethyl, n-propyl or iso-propyl group.

The term “C₁-C₆-haloalkyl” is to be understood as meaning a linear or branched, saturated, monovalent hydrocarbon group in which the term “C₁-C₆-alkyl” is defined supra, and in which one or more hydrogen atoms is replaced by a halogen atom, in identically or differently, i.e. one halogen atom being independent from another. Particularly, said halogen atom is F. Said C₁-C₆-haloalkyl group is, for example, —CF₃, —CHF₂, —CH₂F, —CF₂CF₃ or —CH₂CF₃.

The term “C₁-C₄-hydroxyalkyl” is to be understood as meaning a linear or branched, saturated, monovalent hydrocarbon group in which the term “C₁-C₄-alkyl” is defined supra, and in which one or more hydrogen atoms is replaced by a hydroxy group, e.g. a hydroxymethyl, 1-hydroxyethyl, 2-hydroxyethyl, 1,2-dihydroxyethyl, 3-hydroxypropyl, 2-hydroxypropyl, 2,3-dihydroxypropyl, 1,3-dihydroxypropan-2-yl, 3-hydroxy-2-methyl-propyl, 2-hydroxy-2-methyl-propyl, 1-hydroxy-2-methyl-propyl group.

The term “C₁-C₆-alkoxy” is to be understood as meaning a linear or branched, saturated, monovalent, hydrocarbon group of formula —O-alkyl, in which the term “alkyl” is defined supra, e.g. a methoxy, ethoxy, n-propoxy, iso-propoxy, n-butoxy, iso-butoxy, tert-butoxy, sec-butoxy, pentoxy, iso-pentoxy, or n-hexoxy group, or an isomer thereof. Particularly, said “C₁-C₆-alkoxy” can contain 1, 2, 3, 4 or 5 carbon atoms, (a “C₁-C₅-alkoxy”), preferably 1, 2, 3 or 4 carbon atoms (“C₁-C₄-alkoxy”).

The term “C₁-C₆-haloalkoxy” is to be understood as meaning a linear or branched, saturated, monovalent C₁-C₆-alkoxy group, as defined supra, in which one or more of the hydrogen atoms is replaced, in identically or differently, by a halogen atom. Particularly, said halogen atom is F. Said C₁-C₆-haloalkoxy group is, for example, —OCF₃, —OCHF₂, —OCH₂F, —OCF₂CF₃, or —OCH₂CF₃.

The term “C₂-C₆-alkenyl” is to be understood as meaning a linear or branched, monovalent hydrocarbon group, which contains one or more double bonds, and which has 2, 3, 4, 5 or 6 carbon atoms or 2, 3 or 4 carbon atoms (“C₂-C₄-alkenyl), particularly 2 or 3 carbon atoms (“C₂-C₃-alkenyl”), it being understood that in the case in which said alkenyl group contains more than one double bond, then said double bonds may be isolated from, or conjugated with, each other. Said alkenyl group is, for example, a vinyl, allyl, (E)-2-methylvinyl, (Z)-2-methylvinyl, homoallyl, (E)-but-2-enyl, (Z)-but-2-enyl, (E)-but-1-enyl, (Z)-but-1-enyl, pent-4-enyl, (E)-pent-3-enyl, (Z)-pent-3-enyl, (E)-pent-2-enyl, (Z)-pent-2-enyl, (E)-pent-1-enyl, (Z)-pent-1-enyl, hex-5-enyl, (E)-hex-4-enyl, (Z)-hex-4-enyl, (E)-hex-3-enyl, (Z)-hex-3-enyl, (E)-hex-2-enyl, (Z)-hex-2-enyl, (E)-hex-1-enyl, (Z)-hex-1-enyl, isopropenyl, 2-methylprop-2-enyl, 1-methylprop-2-enyl, 2-methylprop-1-enyl, (E)-1-methylprop-1-enyl, (Z)-1-methylprop-1-enyl, 3-methylbut-3-enyl, 2-methylbut-3-enyl, 1-methylbut-3-enyl, 3-methylbut-2-enyl, (E)-2-methylbut-2-enyl, (Z)-2-methylbut-2-enyl, (E)-1-methylbut-2-enyl, (Z)-1-methylbut-2-enyl, (E)-3-methylbut-1-enyl, (Z)-3-methylbut-1-enyl, (E)-2-methylbut-1-enyl, (Z)-2-methylbut-1-enyl, (E)-1-methylbut-1-enyl, (Z)-1-methylbut-1-enyl, 1,1-dimethylprop-2-enyl, 1-ethylprop-1-enyl, 1-propylvinyl, 1-isopropylvinyl, 4-methylpent-4-enyl, 3-methylpent-4-enyl, 2-methylpent-4-enyl, 1-methylpent-4-enyl, 4-methylpent-3-enyl, (E)-3-methylpent-3-enyl, (Z)-3-methylpent-3-enyl, (E)-2-methylpent-3-enyl, (Z)-2-methylpent-3-enyl, (E)-1-methylpent-3-enyl, (Z)-1-methylpent-3-enyl, (E)-4-methylpent-2-enyl, (Z)-4-methylpent-2-enyl, (E)-3-methylpent-2-enyl, (Z)-3-methylpent-2-enyl, (E)-2-methylpent-2-enyl, (Z)-2-methylpent-2-enyl, (E)-1-methylpent-2-enyl, (Z)-1-methylpent-2-enyl, (E)-4-methylpent-1-enyl, (Z)-4-methylpent-1-enyl, (E)-3-methylpent-1-enyl, (Z)-3-methylpent-1-enyl, (E)-2-methylpent-1-enyl, (Z)-2-methylpent-1-enyl, (E)-1-methylpent-1-enyl, (Z)-1-methylpent-1-enyl, 3-ethylbut-3-enyl, 2-ethylbut-3-enyl, 1-ethylbut-3-enyl, (E)-3-ethylbut-2-enyl, (Z)-3-ethylbut-2-enyl, (E)-2-ethylbut-2-enyl, (Z)-2-ethylbut-2-enyl, (E)-1-ethylbut-2-enyl, (Z)-1-ethylbut-2-enyl, (E)-3-ethylbut-1-enyl, (Z)-3-ethylbut-1-enyl, 2-ethylbut-1-enyl, (E)-1-ethylbut-1-enyl, (Z)-1-ethylbut-1-enyl, 2-propylprop-2-enyl, 1-propylprop-2-enyl, 2-isopropylprop-2-enyl, 1-isopropylprop-2-enyl, (E)-2-propylprop-1-enyl, (Z)-2-propylprop-1-enyl, (E)-1-propylprop-1-enyl, (Z)-1-propylprop-1-enyl, (E)-2-isopropylprop-1-enyl, (Z)-2-isopropylprop-1-enyl, (E)-1-isopropylprop-1-enyl, (Z)-1-isopropylprop-1-enyl, (E)-3,3-dimethylprop-1-enyl, (Z)-3,3-dimethylprop-1-enyl, 1-(1,1-dimethylethyl)ethenyl, buta-1,3-dienyl, penta-1,4-dienyl, hexa-1,5-dienyl, or methylhexadienyl group. Particularly, said group is vinyl or allyl.

The term “C₃-C₁₀-cycloalkyl” is to be understood as meaning a saturated, monovalent, mono-, or bicyclic hydrocarbon ring which contains 3, 4, 5, 6, 7, 8, 9 or 10 carbon atoms (“C₃-C₁₀-cycloalkyl”). Said C₃-C₁₀-cycloalkyl group is for example, a monocyclic hydrocarbon ring, e.g. a cyclopropyl, cyclobutyl, cyclopentyl, cyclohexyl, cycloheptyl, cyclooctyl, cyclononyl or cyclodecyl, or a bicyclic hydrocarbon ring, e.g. a perhydropentalenylene or decalin ring. Particularly, said ring contains 3, 4, 5 or 6 carbon atoms (“C₃-C₆-cycloalkyl”), preferably cyclopropyl.

The term “3- to 10-membered heterocycloalkyl” is to be understood as meaning a saturated, monovalent, mono- or bicyclic hydrocarbon ring which contains 2, 3, 4, 5, 6, 7, 8 or 9 carbon atoms, and one or more heteroatom-containing groups selected from C(═O), O, S, S(═O), S(═O)₂, NRₐ, in which Rₐ represents a hydrogen atom, or a C₁-C₆-alkyl or C₁-C₆-haloalkyl group; it being possible for said heterocycloalkyl group to be attached to the rest of the molecule via any one of the carbon atoms or, if present, the nitrogen atom.

Particularly, said 3- to 10-membered heterocycloalkyl can contain 2, 3, 4, or 5 carbon atoms, and one or more of the above-mentioned heteroatom-containing groups (a “3- to 6-membered heterocycloalkyl”), more particularly said heterocycloalkyl can contain 4 or 5 carbon atoms, and one or more of the above-mentioned heteroatom-containing groups (a “5- to 6-membered heterocycloalkyl”).

Particularly, without being limited thereto, said heterocycloalkyl can be a 4-membered ring, such as an azetidinyl, oxetanyl, or a 5-membered ring, such as tetrahydrofuranyl, dioxolinyl, pyrrolidinyl, imidazolidinyl, pyrazolidinyl, pyrrolinyl, or a 6-membered ring, such as tetrahydropyranyl, piperidinyl, morpholinyl, dithianyl, thiomorpholinyl, piperazinyl, or trithianyl, or a 7-membered ring, such as a diazepanyl ring, for example. Optionally, said heterocycloalkyl can be benzo fused. Preferably, the 3- to 6-membered heterocycloalkyl is a tetrahydrofuranyl, tetrahydropyranyl or piperazinyl.

Said heterocycloalkyl can be bicyclic, such as, without being limited thereto, a 5,5-membered ring, e.g. a hexahydrocyclopenta[c]pyrrol-2(1H)-yl ring, or a 5,6-membered bicyclic ring, e.g. a hexahydropyrrolo[1,2-a]pyrazin-2(1H)-yl ring.

As mentioned supra, said nitrogen atom-containing ring can be partially unsaturated, i.e. it can contain one or more double bonds, such as, without being limited thereto, a 2,5-dihydro-1H-pyrrolyl, 4H-[1,3,4]thiadiazinyl, 4,5-dihydrooxazolyl, or 4H-[1,4]thiazinyl ring, for example, or, it may be benzo-fused, such as, without being limited thereto, a dihydroisoquinolinyl ring, for example.

The term “3- to 10-membered heterocycloalkoxy” of formula —O-heterocycloalkyl, in which the term “heterocycloalkyl” is defined supra, is to be understood as meaning a saturated, monovalent, mono- or bicyclic hydrocarbon ring which contains 2, 3, 4, 5, 6, 7, 8 or 9 carbon atoms, and one or more heteroatom-containing groups selected from C(═O), O, S, S(═O), S(═O)₂, NRₐ, in which Rₐ represents a hydrogen atom, a C₁-C₆-alkyl or C₁-C₆-haloalkyl group and which is connected to the rest of the molecule via an oxygen atom, e.g. a pyrrolidineoxy, tetrahydrofuraneoxy or tetrahydropyranoxy.

The term “4- to 10-membered heterocycloalkenyl” is to be understood as meaning an unsaturated, monovalent, mono- or bicyclic hydrocarbon ring which contains 3, 4, 5, 6, 7, 8 or 9 carbon atoms, and one or more heteroatom-containing groups selected from C(═O), O, S, S(═O), S(═O)₂, NRₐ, in which Rₐ represents a hydrogen atom, or a C₁-C₆-alkyl or C₁-C₆-haloalkyl group; it being possible for said heterocycloalkenyl group to be attached to the rest of the molecule via any one of the carbon atoms or, if present, the nitrogen atom. Examples of said heterocycloalkenyl may contain one or more double bonds, e.g. 4H-pyranyl, 2H-pyranyl, 3,6-dihydro-2H-pyran-4-yl, 3,6-dihydro-2H-thiopyran-4-yl, 1,2,3,6-tetrahydropyridin-4-yl, 3H-diazirinyl, 2,5-dihydro-1H-pyrrolyl, [1,3]dioxolyl, 4H-[1,3,4]thiadiazinyl, 2,5-dihydrofuranyl, 2,3-dihydrofuranyl, 2,5-dihydrothiophenyl, 2,3-dihydrothiophenyl, 4,5-dihydrooxazolyl, 4H-[1,4]thiazinyl or 5,6-dihydroimidazo[1,2-a]pyrazin-7(8H)-yl group or it may be benzo fused.

The term “heteroaryl” is understood as meaning a monovalent, monocyclic-, bicyclic- or tricyclic aromatic ring system having 5, 6, 7, 8, 9, 10, 11, 12, 13 or 14 ring atoms (a “5- to 14-membered heteroaryl” group), 5 or 6 or 9 or 10 ring atoms (a “5- to 10-membered heteroaryl” group) or particularly 5 or 6 ring atoms (“5- to 6-membered heteroaryl” group), and which contains at least one heteroatom which may be identical or different, said heteroatom being such as oxygen, nitrogen or sulfur, and in addition in each case can be benzocondensed. Particularly, heteroaryl is selected from thienyl, furanyl, pyrrolyl, oxazolyl, thiazolyl, imidazolyl, pyrazolyl, isoxazolyl, isothiazolyl, oxadiazolyl, triazolyl, thiadiazolyl, thia-4H-pyrazolyl etc., and benzo derivatives thereof, such as, for example, benzofuranyl, benzothienyl, benzoxazolyl, benzisoxazolyl, benzimidazolyl, benzotriazolyl, indazolyl, indolyl, isoindolyl, etc.; or pyridyl, pyridazinyl, pyrimidinyl, pyrazinyl, triazinyl, etc., and benzo derivatives thereof, such as, for example, quinolinyl, quinazolinyl, isoquinolinyl, etc.; or azocinyl, indolizinyl, purinyl, etc., and benzo derivatives thereof; or cinnolinyl, phthalazinyl, quinazolinyl, quinoxalinyl, naphthpyridinyl, pteridinyl, carbazolyl, acridinyl, phenazinyl, phenothiazinyl, phenoxazinyl, xanthenyl, oxepinyl or 1H-pyrrolo[2,3-b]pyridin-4-yl, etc.

In general, and unless otherwise mentioned, the heteroarylic or heteroarylenic radicals include all the possible isomeric forms thereof, e.g. the positional isomers thereof. Thus, for some illustrative non-restricting example, the term pyridinyl or pyridinylene includes pyridin-2-yl, pyridin-2-ylene, pyridin-3-yl, pyridin-3-ylene, pyridin-4-yl and pyridin-4-ylene; or the term thienyl or thienylene includes thien-2-yl, thien-2-ylene, thien-3-yl and thien-3-ylene.

The term “C₁-C₆”, as used throughout this text, e.g. in the context of the definition of “C₁-C₆-alkyl”, “C₁-C₆-haloalkyl”, “C₁-C₆-alkoxy”, or “C₁-C₆-haloalkoxy” is to be understood as meaning an alkyl group having a finite number of carbon atoms of 1 to 6, i.e. 1, 2, 3, 4, 5, or 6 carbon atoms. It is to be understood further that said term “C₁-C₆” is to be interpreted as any sub-range comprised therein, e.g. C₁-C₆, C₂-C₅, C₃-C₄, C₁-C₂, C₁-C₃, C₁-C₄, C₁-C₅; particularly C₁-C₂, C₁-C₃, C₁-C₄, C₁-C₅, C₁-C₆; more particularly C₁-C₄; in the case of “C₁-C₆-haloalkyl” or “C₁-C₆-haloalkoxy” even more particularly C₁-C₂.

Similarly, as used herein, the term “C₂-C₆”, as used throughout this text, e.g. in the context of the definitions of “C₂-C₆-alkenyl” and “C₂-C₆-alkynyl”, is to be understood as meaning an alkenyl group or an alkynyl group having a finite number of carbon atoms of 2 to 6, i.e. 2, 3, 4, 5, or 6 carbon atoms. It is to be understood further that said term “C₂-C₆” is to be interpreted as any sub-range comprised therein, e.g. C₂-C₆, C₃-C₅, C₃-C₄, C₂-C₃, C₂-C₄, C₂-C₅; particularly C₂-C₃.

Further, as used herein, the term “C₃-C₆”, as used throughout this text, e.g. in the context of the definition of “C₃-C₆-cycloalkyl”, is to be understood as meaning a cycloalkyl group having a finite number of carbon atoms of 3 to 6, i.e. 3, 4, 5 or 6 carbon atoms. It is to be understood further that said term “C₃-C₆” is to be interpreted as any sub-range comprised therein, e.g. C₃-C₆, C₄-C₅, C₃-C₅, C₃-C₄, C₄-C₆, C₅-C₆; particularly C₃-C₆.

Further, as used herein, the term “C₂-C₄”, as used throughout this text, e.g. in the context of “C₂-C₄-alkenyl” is to be understood as meaning a alkenyl group having a finite number of carbon atoms of 2 to 4, i.e. 2, 3 or 4 carbon atoms. It is to be understood further that said term “C₂-C₄” is to be interpreted as any sub-range comprised therein, e.g. C₂-C₄, C₂-C₃, C₃-C₄.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds.

The term “optionally substituted” means optional substitution with the specified groups, radicals or moieties.

Ring system substituent means a substituent attached to an aromatic or nonaromatic ring system which, for example, replaces an available hydrogen on the ring system.

By “stable compound’ or “stable structure” is meant a compound that is sufficiently robust to survive isolation to a useful degree of purity from a reaction mixture, and formulation into an efficacious therapeutic agent.

As used herein, the term “one or more”, e.g. in the definition of the substituents of the compounds of the general formulae of the present invention, is understood as meaning “one, two, three, four or five, particularly one, two, three or four, more particularly one, two or three, even more particularly one or two”.

The invention also includes all suitable isotopic variations of the compound of component A. An isotopic variation of the compound of component A is defined as one in which at least one atom is replaced by an atom having the same atomic number but an atomic mass different from the atomic mass usually or predominantly found in nature. Examples of isotopes that can be incorporated into the compound of component A include isotopes of hydrogen, carbon, nitrogen, oxygen, phosphorus, sulphur, fluorine, chlorine, bromine and iodine, such as ²H (deuterium), ³H (tritium), ¹¹C, ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁹I and ¹³¹I, respectively. Certain isotopic variations of the compound of component A, for example, those in which one or more radioactive isotopes such as ³H or ¹⁴C are incorporated, are useful in drug and/or substrate tissue distribution studies. Tritiated and carbon-14, i.e., ¹⁴C, isotopes are particularly preferred for their ease of preparation and detectability. Further, substitution with isotopes such as deuterium may afford certain therapeutic advantages resulting from greater metabolic stability, for example, increased in vivo half-life or reduced dosage requirements and hence may be preferred in some circumstances. Isotopic variations of the compound of component A can generally be prepared by conventional procedures known by a person skilled in the art such as by the illustrative methods or by the preparations described in the examples hereafter using appropriate isotopic variations of suitable reagents.

Where the plural form of the word compounds, salts, polymorphs, hydrates, solvates and the like, is used herein, this is taken to mean also a single compound, salt, polymorph, isomer, hydrate, solvate or the like.

The compounds of component A may contain one or more asymmetric centre, depending upon the location and nature of the various substituents desired. Asymmetric carbon atoms may be present in the (R) or (S) configuration, resulting in racemic mixtures in the case of a single asymmetric centre, and diastereomeric mixtures in the case of multiple asymmetric centres. In certain instances, asymmetry may also be present due to restricted rotation about a given bond, for example, the central bond adjoining two substituted aromatic rings of the specified compounds. The compounds of component A may contain sulphur atoms which are asymmetric, such as an asymmetric sulphoxide or sulphoximine group, of structure:

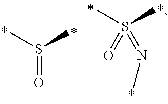

for example, in which * indicates atoms to which the rest of the molecule can be bound.

Substituents on a ring may also be present in either cis or trans form. It is intended that all such configurations (including enantiomers and diastereomers), are included within the scope of the present invention.

Preferred compounds of component A are those which produce the more desirable biological activity. Separated, pure or partially purified isomers and stereoisomers or racemic or diastereomeric mixtures of the compounds of component A are also included within the scope of the present invention. The purification and the separation of such materials can be accomplished by standard techniques known in the art.

The optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, ditoluoyltartaric and camphorsulfonic acid. Mixtures of diastereoisomers can be separated into their individual diastereomers on the basis of their physical and/or chemical differences by methods known in the art, for example, by chromatography or fractional crystallisation. The optically active bases or acids are then liberated from the separated diastereomeric salts. A different process for separation of optical isomers involves the use of chiral chromatography (e.g., chiral HPLC columns), with or without conventional derivatisation, optimally chosen to maximise the separation of the enantiomers. Suitable chiral HPLC columns are manufactured by Daicel, e.g., Chiracel OD and Chiracel OJ among many others, all routinely selectable. Enzymatic separations, with or without derivatisation, are also useful. The optically active compounds of this invention can likewise be obtained by chiral syntheses utilizing optically active starting materials.

In order to limit different types of isomers from each other reference is made to IUPAC Rules Section E (Pure Appl Chem 45, 11-30, 1976).

The present invention includes all possible stereoisomers of the compounds of component A as single stereoisomers, or as any mixture of said stereoisomers, e.g. R- or S-isomers, or E- or Z-isomers, in any ratio. Isolation of a single stereoisomer, e.g. a single enantiomer or a single diastereomer, of a compound of component A may be achieved by any suitable state of the art method, such as chromatography, especially chiral chromatography, for example.

Further, the compounds of component A, particularly Compound A, may exist as tautomers. For example, any compound of component A which contains a pyrazole moiety as a heteroaryl group for example can exist as a 1H tautomer, or a 2H tautomer, or even a mixture in any amount of the two tautomers, or a triazole moiety for example can exist as a 1H tautomer, a 2H tautomer, or a 4H tautomer, or even a mixture in any amount of said 1H, 2H and 4H tautomers, namely:

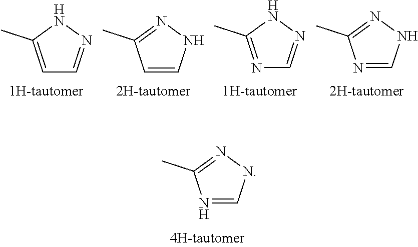

The present combination includes all possible tautomers of the compounds of component A, particularly the 1H-tautomer or the 2H-tautomer of the pyrazol-5-yl group in 8-position of the naphthyridine core of Compound A, as single tautomers, or as any mixture of said tautomers, in any ratio.

Further, the compounds of component A can exist as N-oxides, which are defined in that at least one nitrogen of the compounds of the present invention is oxidised. The present combination includes all such possible N-oxides of component A.

The present combination also relates to useful forms of the compounds as disclosed herein, such as metabolites, hydrates, solvates, prodrugs, salts, in particular pharmaceutically acceptable salts, and co-precipitates.

The compounds of the present combination can exist as a hydrate, or as a solvate, wherein the compounds of the present combination contain polar solvents, in particular water, methanol or ethanol for example as structural element of the crystal lattice of the compounds. The amount of polar solvents, in particular water, may exist in a stoichiometric or non-stoichiometric ratio. In the case of stoichiometric solvates, e.g. a hydrate, hemi-, (semi-), mono-, sesqui-, di-, tri-, tetra-, penta-etc. solvates or hydrates, respectively, are possible. The present combination includes all such hydrates or solvates.

Further, the compounds of the present combination can exist in free form, e.g. as a free base, or as a free acid, or as a zwitterion, or can exist in the form of a salt. Said salt may be any salt, either an organic or inorganic addition salt, particularly any pharmaceutically acceptable organic or inorganic addition salt, customarily used in pharmacy.

The present invention includes all possible salts of the components of the present combination as single salts, or as any mixture of said salts, in any ratio.

Furthermore, the present invention includes all possible crystalline forms, or polymorphs, of the compounds of components of the present combination, either as single polymorphs, or as a mixture of more than one polymorph, in any ratio.

When radicals in the compounds of the present combination are substituted, the radicals may be mono- or polysubstituted, unless specified otherwise. In the context of the present invention, all radicals which occur more than once are defined independently of one another. Substitution by one, two or three identical or different substituents is preferred.

In the context of the present invention, the term “treatment” or “treating” includes inhibition, retardation, checking, alleviating, attenuating, restricting, reducing, suppressing, repelling or healing of a disease or the development, the course or the progression of such states and/or the symptoms of such states. The term “disease” includes but is not limited a condition, a disorder, an injury or a health problem. The term “therapy” is understood here to be synonymous with the term “treatment”.

The terms “prevention”, “prophylaxis” or “preclusion” are used synonymously in the context of the present invention and refer to the avoidance or reduction of the risk of contracting, experiencing, suffering from or having a disease or a development or advancement of such states and/or the symptoms of such states.

The treatment or prevention of a disease may be partial or complete.

### Component A of the Combination

Component A can be Selected from Radioactive Nuclei

The tissue-targeting radiopharmaceutical preferably comprises an alpha-emitter. The radioactive isotope may be any alpha-emitting isotope (i.e. an alpha emitter) suitable for use in the treatments of the present invention. The alpha emitters may be selected from the group consisting of Terbium-149 (¹⁴⁹Tb), Astatine-211 (²¹¹At), Bismuth-212 (²¹²Bi), Bismuth-213 (²¹³Bi), Actinium-225 (²²⁵Ac), or Thorium-227 (²²⁷Th). Preferably, the alpha-emitting nucleus is Thorium-227.

In a particular embodiment of the invention the tissue-targeting radiopharmaceutical is a complex comprising the 4+ ion of an alpha emitting thorium radionuclide, such as Thorium-227. Preferably, the tissue-targeting radiopharmaceutical is a targeted thorium conjugate (TTC). The targeted thorium conjugate may be any conjugate which comprises an alpha-radioactive thorium ion (e.g. Thorium-227 ion) linked to a targeting moiety such as those described previously. In particular, preferred targeted thorium conjugates is PSMA-TTC.

Radioactive thorium-containing compounds (e.g. comprising Th-227) may be used in high dose regimens, where the myelotoxicity of the generated radium (e.g. Ra-223) would normally be intolerable, when stem cell support or a comparable recovery method is included. Without supportive intervention, the maximum dose of a nuclide such as ²²⁷Th may be limited by such myelotoxicity and might be stopped, for example, to avoid depressing the neutrophil cell count below 20% or 10% of its initial value at nadir. In cases of stem-cell support or similar supportive therapy is provided, the neutrophil cell count may be reduced to below 10% at nadir and exceptionally will be reduced to 5% or if necessary below 5%, providing suitable precautions are taken and subsequent stem cell support is given. Such techniques are well known in the art.

Alpha-emitting thorium is the preferred radioactive element comprised in the tissue-targeting radiopharmaceuticals referred to herein and Thorium-227 is the preferred isotope for all references to thorium herein where context allows. Thorium-227 is relatively easy to produce and can be prepared indirectly from neutron irradiated Ra-226, which will contain the mother nuclide of Th-227, i.e. Ac-227 (T1/2=22 years). Actinium-227 can quite easily be separated from the Ra-226 target and used as a generator for Th-227. This process can be scaled to industrial scale if necessary, and hence the supply problem seen with most other alpha-emitters considered candidates for molecular targeted radiotherapy can be avoided. Thorium-227 decays via radium-223. In this case the primary daughter has a half-life of 11.4 days. From a pure Th-227 source, only moderate amounts of radium are produced during the first few days. However, the potential toxicity of Ra-223 is higher than that of Th-227 since the emission from Ra-223 of an alpha particle is followed within minutes by three further alpha particles from the short-lived daughters.

Partly because it generates potentially harmful decay products, thorium-227 (T1/2=18.7 days) has not been widely considered for alpha particle therapy.

Thorium-227 may be administered in amounts sufficient to provide desirable therapeutic effects without generating so much radium-223 as to cause intolerable bone marrow suppression. It is desirable to maintain the daughter isotopes in the targeted region so that further therapeutic effects may be derived from their decay. However, it is not necessary to maintain control of the thorium decay products in order to have a useful therapeutic effect without inducing unacceptable myelotoxicity. Without being bound by theory, this is believed to be because at least partial incorporation of the radium-223 into bone and the short half-life of the daughters serves to titrate the potentially harmful daughter nuclei away from sensitive structures such as the bone marrow.

The alpha-emitting isotope of the radiopharmaceutical may be linked to the tissue-targeting moiety via any suitable ligand. Such a ligand will be selected to be appropriate for the chemistry of the relevant element and oxidation state and suitable chelators are generally well-known in the art.

Previously known chelators for thorium, for example, include the polyaminopolyacid chelators which comprise a linear, cyclic or branched polyazaalkane backbone with acidic (e.g. carboxyalkyl) groups attached at backbone nitrogens. Examples of such chelators include DOTA derivatives such as p-isothiocyanatobenzyl-1,4,7,10-tetraazacyclododecane-1,4,7,10-tetraacetic acid (p-SCN-Bz-DOTA) and DTPA derivatives such as p-isothiocyanatobenzyl-diethylenetriaminepentaacetic acid (p-SCN-Bz-DTPA), the first being cyclic chelators, the latter linear chelators.

In one particular embodiment of the invention, the tissue-targeting radiopharmaceutical comprises a tissue-targeting moiety covalently bound to an octadentate ligand, examples of which include ligands comprising at least one 3,2-hydroxypyridinone (3,2-HOPO) moiety. Said ligand may be complexed to a 4+ metal ion such as that of and alpha-emitting thorium radionuclide (e.g. ²²⁷Th). Such ligands are described, for example, in WO2011/098611 which is incorporated herein by reference. The ligand may therefore be an octadentate ligand, particularly an octadentate hydroxypyridinone-containing ligand. Such ligands will typically comprise at least one chelating group of the following substituted pyridine structure (I):

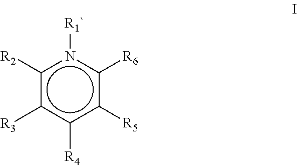

Wherein R₁ is an optional N-substituent group and may thus be absent or may be selected from hydrocarbyl, OH, O-hydrocarbyl, SH and S-hydrocarbyl groups (e.g. methyl or ethyl); comprises a linker moiety; and/or comprises a coupling moiety; groups R₂ to R₆ are each independently selected from H, OH, ═O, short hydrocarbyl groups (e.g. methyl, ethyl, propyl), linker moieties (linking to other moieties of formula I) and/or coupling moieties (coupling to targeting agents). Favoured ligands may have four moieties of formula I as described in WO2011/098611. Particular examples include octadentate 3,2-HOPO ligands such as those indicated below, as well as equivalent ligands additionally substituted with linker groups (if needed), as discussed herein:

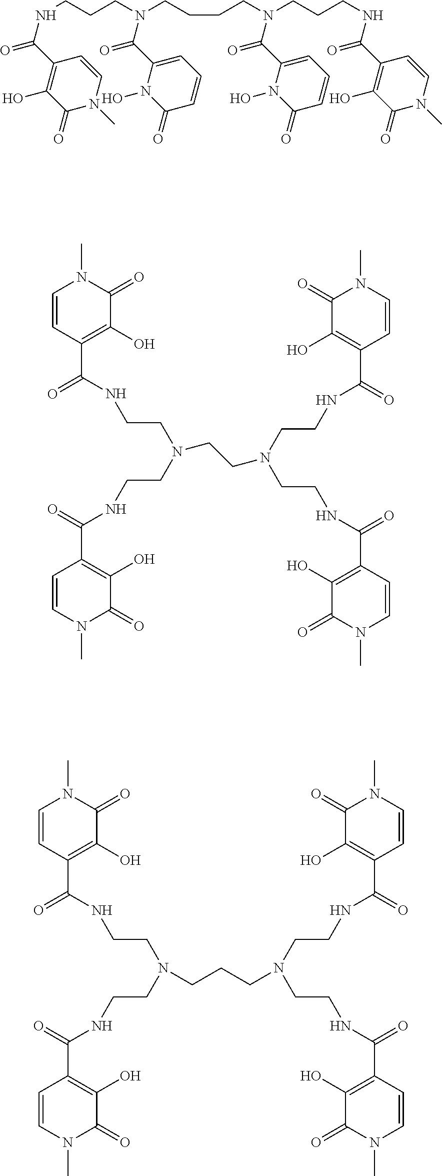 [SMILES: Cn1ccc(C(=O)NCCCN(CCCCN(CCCNC(=O)c2ccn(C)c(=O)c2O)C(=O)c2cccc(=O)n2O)C(=O)c2cccc(=O)n2O)c(O)c1=O ; Cn1ccc(C(=O)NCCN(CCNC(=O)c2ccn(C)c(=O)c2O)CCN(CCNC(=O)c2ccn(C)c(=O)c2O)CCNC(=O)c2ccn(C)c(=O)c2O)c(O)c1=O ; Cn1ccc(C(=O)NCCN(CCCN(CCNC(=O)c2ccn(C)c(=O)c2O)CCNC(=O)c2ccn(C)c(=O)c2O)CCNC(=O)c2ccn(C)c(=O)c2O)c(O)c1=O]

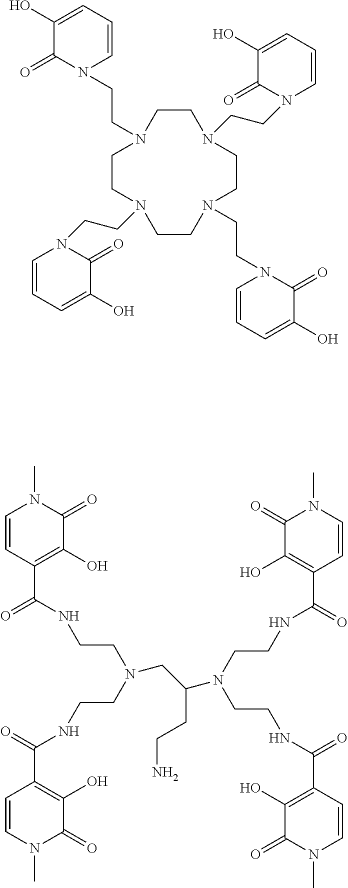 [SMILES: O=c1c(O)cccn1CCN1CCN(CCn2cccc(O)c2=O)CCN(CCn2cccc(O)c2=O)CCN(CCn2cccc(O)c2=O)CC1 ; Cn1ccc(C(=O)NCCN(CCNC(=O)c2ccn(C)c(=O)c2O)CC(CCN)N(CCNC(=O)c2ccn(C)c(=O)c2O)CCNC(=O)c2ccn(C)c(=O)c2O)c(O)c1=O]

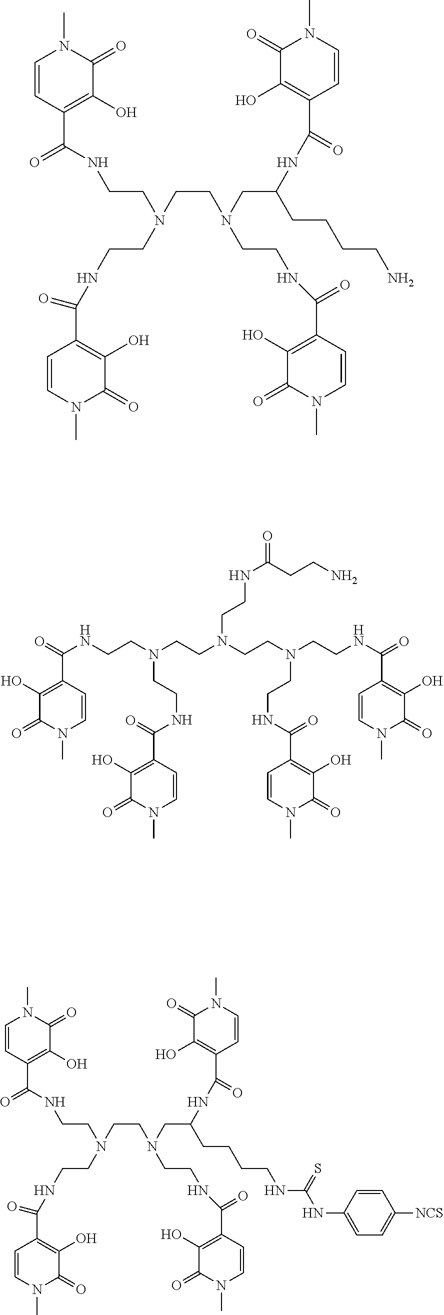

An alternative favoured embodiment utilises ligands as described in WO2013/167756, which is incorporated herein by reference. Such ligands may also be complexed to a 4+ metal ion such as that of an alpha-emitting thorium radionuclide (e.g. ²²⁷Th). In such a particular embodiment, the ligand can be an octadentate ligand comprising at least one and preferably two or four chelating moieties of formula II:

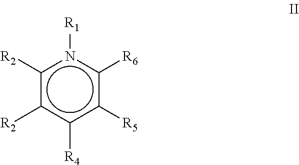

Wherein R₁ is an optional N-substituent solubilising group which will be present in at least one of the moieties of formula II (e.g. in 1 to 4 of four moieties of formula II) and comprises a hydroxyalkyl group (e.g. hydroxymethyl or hydroxydethyl group); groups R₂ to R₆ are each independently selected from H, OH, ═O, short hydrocarbyl groups, linker moieties and/or coupling moieties wherein one of R₂ to R₆ is OH and one of R₂ to R₆ is ═O. The remaining groups R₂ to R₆ may be as described above. The ligand may for example be a ligand of structure III:

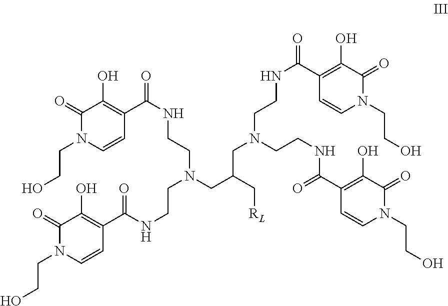

Wherein R_{L} is any suitable linker moiety such as -Ph-NH₂, -Ph-NCS, -Ph-NH—CO—C₂H₄—CO₂H or any described herein.

As used herein, the term “linker moiety” is used to indicate a chemical entity which serves to join at least two chelating groups in the octadentate ligands, which form a key component in various aspects of the invention. Typically, each chelating group (e.g. those of formula I above and/or formula II below) will be bi-dentate and so four chelating groups, of which at least one is of formula I, will typically be present in the ligand. Such chelating groups are joined to each other by means of their linker moieties. Thus, a linker moiety (as used above) may be shared between more than one chelating group of formula I and/or II. The linker moieties may also serve as the point of attachment between the complexing part and the targeting moiety. In such a case, at least one linker moiety will join to a coupling moiety (see below). Suitable linker moieties include short hydrocarbyl groups, such as C1 to C12 hydrocarbyl, including C1 to C12 alkyl, alkenyl or alkynyl group, including methyl, ethyl, propyl, butyl, pentyl and/or hexyl groups of all topologies.

Linker moieties may also be or comprise any other suitably robust chemical linkages including esters, ethers, amine and/or amide groups. The total number of atoms joining two chelating moieties (counting by the shortest path if more than one path exists) will generally be limited, so as to constrain the chelating moieties in a suitable arrangement for complex formation. Thus, linker moieties will typically be chosen to provide no more than 15 atoms between chelating moieties, preferably, 1 to 12 atoms, and more preferably 1 to 10 atoms between chelating moieties. Where a linker moiety joins two chelating moieties directly, the linker will typically be 1 to 12 atoms in length, preferably 2 to 10 (such as ethyl, propyl, n-butyl etc). Where the linker moiety joins to a central template (see below) then each linker may be shorter with two separate linkers joining the chelating moieties. A linker length of 1 to 8 atoms, preferably 1 to 6 atoms may be preferred in this case (methyl, ethyl and propyl being suitable, as are groups such as these having an ester, ether or amide linkage at one end or both).

A “coupling moiety” as used herein serves to link the ligand component (e.g. with 4 moieties of formula I and/or II) to the targeting moiety. Preferably coupling moieties will be covalently linked to the chelating groups, either by direct covalent attachment to one of the chelating groups or more typically by attachment to a linker moiety or template. Should two or more coupling moieties be used, each can be attached to any of the available sites such as on any template, linker or chelating group.

In one embodiment, the coupling moiety may have the structure:

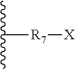

wherein R₇ is a bridging moiety, which is a member selected from substituted or unsubstituted alkyl, substituted or unsubstituted heteroalkyl, substituted or unsubstituted heterocycloalkyl, substituted or unsubstituted aryl and substituted or unsubstituted heteroaryl; and X is a targeting moiety or a reactive functional group. The preferred bridging moieties include all those groups indicated herein as suitable linker moieties. Preferred targeting moieties include all of those described herein and preferred reactive X groups include any group capable of forming a covalent linkage to a targeting moiety, including, for example, COOH, OH, SH, NHR and COH groups, where the R of NHR may be H or any of the short hydrocarbyl groups described herein. Highly preferred groups for attachment onto the targeting moiety include epsilon-amines of lysine residues and thiol groups of cysteine residues. Non-limiting examples of suitable reactive X groups, include N-hydroxysuccimidylesters, imidoesters, acylhalides, N-maleimides, alpha-halo acetyl and isothiocyanates, where the latter three are suitable for reaction with a thiol group.

Another typical example of an octadentate chelator suitable for use in the present invention is the compound of formula IV below, which utilises the 3-hydroxy-N-methyl-2-pyridinone moiety, abbreviated as Me-3,2-HOPO.

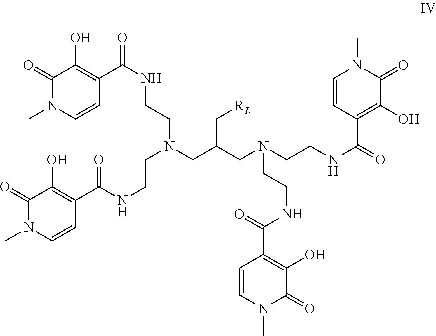

In a particularly favoured embodiment, R_{L} may be such that formula IV is the compound of formula IV′:

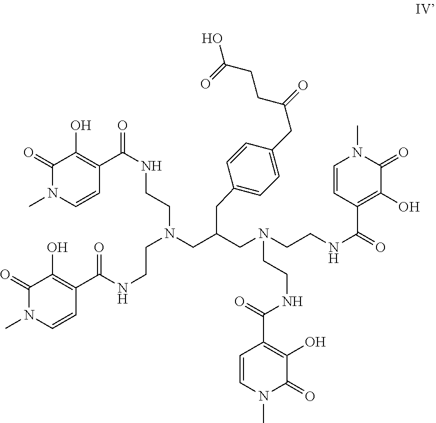 [SMILES: Cn1ccc(C(=O)NCCN(CCNC(=O)c2ccn(C)c(=O)c2O)CC(Cc2ccc(CC(=O)CCC(=O)O)cc2)CN(CCNC(=O)c2ccn(C)c(=O)c2O)CCNC(=O)c2ccn(C)c(=O)c2O)c(O)c1=O]

This particular chelator (IV′) has been found to complex Th-227 in near quantitative yield at ambient temperature in aqueous solutions, and the resulting complexes are highly stable. The carboxylic acid group facilitates conjugation to biomolecules such as antibodies. The synthesis, labelling and in vivo distribution in mice are described in: Bioorganic & Medicinal Chemistry Letters 26 (2016) 4318-4321. It has been shown that the above compound IV′ outperforms 1,4,7,10-tetraazacycloododecane-N, N′,N″,N′″-tetraacetic acid (DOTA) in Th-227 complexation.

In one embodiment, PSMA-TTC is BAY 2315497 and is prepared according to Example 9, specifically Examples 9a and 9b of WO 2016/096843. The monoclonal antibody may be AB-PG1-XG1-006 as disclosed in WO 03/034903.

In all aspects of the present invention, the tissue-targeting radiopharmaceutical preferably comprises Th-227. The radiopharmaceutical is preferably administered at a dosage level of thorium-227 dosage of 18 to 400 kBq/kg bodyweight, preferably 20 to 200 kBq/kg, (such as 50 to 200 kBq/kg) more preferably 75 to 170 kBq/kg, especially 100 to 130 kBq/kg. Correspondingly, a single dosage until may comprise around any of these ranges multiplied by a suitable bodyweight, such as 30 to 150 Kg, preferably 40 to 100 Kg (e.g. a range of 540 kBq to 4000 KBq per dose etc). The thorium dosage, the complexing agent and the administration route will moreover desirably be such that the radium-223 dosage generated in vivo is less than 300 kBq/kg, more preferably less than 200 kBq/kg, still more preferably less than 150 kBq/kg, especially less than 100 kBq/kg. Again, this will provide an exposure to Ra-223 indicated by multiplying these ranges by any of the bodyweights indicated. The above dose levels are preferably the fully retained dose of Th-227 but may be the administered dose taking into account that some Th-227 will be cleared from the body before it decays.

Component A may be administered by the oral, intravenous, topical, local installations, intraperitoneal or nasal route.

Component A may be in the form of a pharmaceutical formulation which is ready for use to be administered simultaneously, concurrently, separately or sequentially with component B and optionally component C as further described infra. The components A and B and optionally C may be administered independently of one another by the oral, intravenous, topical, local installations, intraperitoneal or nasal route.

### Component B of the Combination

Component B of the combination of the present invention is an antiandrogen, particularly an AR antagonist selected from cyproterone acetate, bicalutamide, flutamide, nilutamide, enzalutamide, apalutamide, darolutamide or keto-darolutamide, or an AR degrader such as ARV-110, or an AR N-terminal domain binder such as EPI-506, or an antisense oligonucleotide that reduces AR expression such as EZN-4176 or AZD-5312, or an androgen synthesis inhibitor such as abiraterone, particularly abiraterone acetate, seviteronel, galeterone, orteronel or ketoconazole, or a dual AR antagonist and CYP17A inhibitor such as ODM-204.

According to another embodiment of the aspects of the present invention, component B is an antiandrogen selected from bicalutamide, enzalutamide, apalutamide, darolutamide and abiraterone, particularly abiraterone acetate.

According to a preferred embodiment of the aspects of the present invention, component B is an AR antagonist selected from enzalutamide and darolutamide.

AR antagonists compete with the natural androgens such as testosterone and its more active metabolite dihydrotestosterone (DHT) for binding to the AR in the prostate gland and in other tissues.

Cyproterone acetate (abbreviated as CPA) is an AR antagonist and progestin that is used in the treatment of androgen-related conditions like acne, hirsutism, early-onset puberty, and prostate cancer, as a component of hormone therapy for transgender women, and in oral contraceptives [F. Neumann, J. Kalmus: Cyproterone acetate in the treatment of sexual disorders: pharmacological base and clinical experience. Exp. Clin. Endocrinol. 98, 71-80, 1991].

Bicalutamide is a non-steroidal AR antagonist that is primarily used to treat castration-resistant prostate cancer (CRPC). It is typically used after androgen deprivation therapy by a gonadotropin-releasing hormone (GnRH) analogue or by surgical removal of the testicles to treat metastatic CRPC [Y. Fradet: Bicalutamide (Casodex) in the treatment of prostate cancer. Expert Rev. Anticancer 4, 37-48, 2004].

Flutamide is a non-steroidal AR antagonist used primarily to treat metastatic CRPC [R. N. Brogden, P. Chrisp: Flutamide. A review of its pharmacodynamic and pharmacokinetic properties, and therapeutic use in advanced prostatic cancer. Drugs Aging 1, 104-115, 1991].

Nilutamide is a non-steroidal AR antagonist used in the treatment of metastatic CRPC [E. J. Dole, M T Holdsworth: Nilutamide: an antiandrogen for the treatment of prostate cancer. Ann. Pharmacother. 31, 65-75, 1997].

Enzalutamide is a second-generation AR antagonist used to treat metastatic CRPC [R. M. Bambury, H. I. Scher: Enzalutamide: Development from bench to bedside. Urol. Oncol. 33, 280-288, 2015]. Enzalutamide may also be effective in the treatment of certain types of breast cancer [A. Gucalp, T. A. Traina:Targeting the androgen receptor in triple-negative breast cancer. Curr. Probl. Cancer 40, 141-150, 2016].

Apalutamide (developmental code name ARN-509, also JNJ-56021927) is a second-generation AR antagonist that is under clinical development for the treatment of prostate cancer [D. E. Rathkopf, E. S. Antonarakis et al.: Safety and antitumor activity of apalutamide (ARN-509) in metastatic castration-resistant prostate cancer with and without prior abiraterone acetate and prednisone. Clin. Cancer Res. DOI: 10.1158/1078-0432.CCR-16-2509, 2017].

Darolutamide (also known as ODM-201, BAY 1841788 or N-((S)-1-(3-(3-Chloro-4-cyanophenyl)-1H-pyrazol-1-yl-)-propan-2-yl)-5-(1-hydroxyethyl)-1H-pyrazole-3-carboxamide) is a non-steroidal AR antagonist that has demonstrated significant antitumor activity in different prostate cancer models. It is a mixture of two diastereomers ORM 16555 (also known (S,S)-darolutamide and as N-{(2S)-1-[3-(3-chloro-4-cyanophenyl)-1H-pyrazol-1-yl]propan-2-yl}-5-[(1S)-1-hydroxyethyl]-1H-pyrazole-3-carboxamide) and ORM 16497 (also known as (S,R)-darolutamide and as N-{(2S)-1-[3-(3-chloro-4-cyanophenyl)-1H-pyrazol-1-yl]propan-2-yl}-5-[(1R)-1-hydroxyethyl]-1H-pyrazole-3-carboxamide). ORM-15341 (also known as keto-darolutamide and as 5-acetyl-N-{(2S)-1-[3-(3-chloro-4-cyanophenyl)-1H-pyrazol-1-yl]propan-2-yl}-1H-pyrazole-3-carboxamide) is the main metabolite of darolutamide and possesses similar pharmacological properties [A. J. Moilanen, R Riikonen, et al.: Discovery of ODM-201, a new generation androgen receptor inhibitor targeting resistance mechanisms to androgen signaling-directed prostate cancer therapies. Sci. Rep. 5, 12007, 2005].

Darolutamide (ODM-201) is of structure

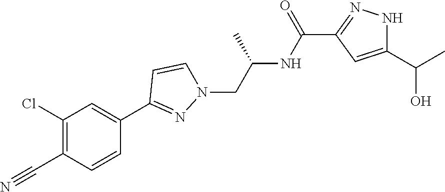 [SMILES: CC(O)c1cc(C(=O)N[C@@H](C)Cn2ccc(-c3ccc(C#N)c(Cl)c3)n2)n[nH]1]

Keto-darolutamide (ORM-15341) is of structure:

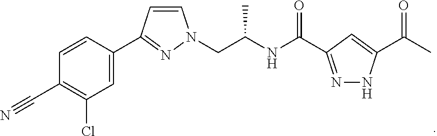 [SMILES: CC(=O)c1cc(C(=O)N[C@@H](C)Cn2ccc(-c3ccc(C#N)c(Cl)c3)n2)n[nH]1]

EPI-506 is a non-steroidal antiandrogen in clinical trials for prostate cancer [G. Martinez-Ariza, C. Hulme: Recent advances in allosteric androgen receptor inhibitors for the potential treatment of castration-resistant prostate cancer. Pharm. Pat. Anal. 4, 387-402, 2015]. It is the successor of EPI-001 and targets the N-terminal domain of the androgen receptor. This mechanism of action is believed to allow the drug to block signaling from the AR and its splice variants. EPI-506 is a prodrug of EPI-002 [Y. Imamura, M. D. Sadar: Androgen receptor targeted therapies in castration-resistant prostate cancer. Int. J. Urol. 23, 654-665, 2016], one of the four stereoisomers of EPI-001 [J. K. Myung, C. Banuelos et al.: An androgen receptor N-terminal domain antagonist for treating prostate cancer. J. Clin. Invest., 123, 2948-2960, 2013].

ARV-110 is an oral AR degrader that is planned to enter clinical phase 1 in early 2019 for the indication metastatic CRPC (http://ir.arvinas.com/news-releases/news-release-details/arvinas-receives-authorization-proceed-its-ind-application).

EZN-4176 is a nucleic acid antisense oligonucleotide directed against exon 4 of the AR gene which was evaluated in a phase 1a/1b clinical study in CRPC patients (Bianchini et al., Br J Cancer 2013, DOI: 10.1038/bjc.2013.619).

AZD-5312 (also named ARRx) is an antisense oligonucleotide targeting the AR which was evaluated in phase 1 dose-escalation study in patients with prostate cancer (Dellis et al. Expert Opin Pharmacother 2018, DOI: 10.1080/14656566.2018.1548611).

Different blockers of androgen synthesis have been described. They inhibit the enzyme CYP17A1 which is expressed in testicular, adrenal, and prostatic tumor tissues. CYP17 catalyzes two sequential reactions: (a) the conversion of pregnenolone and progesterone to their 17α-hydroxy derivatives by its 17α-hydroxylase activity, and (b) the subsequent formation of dehydroepiandrosterone (DHEA) and androstenedione, respectively, by its 17,20-lyase activity [D. Poubek: CYP17A1: a biochemistry, chemistry, and clinical review. Curr. Top. Med. Chem. 13, 1364-1384, 2013]. DHEA and androstenedione are precursors of the more potent androgens testosterone and dihydrotestosterone. Inhibition of CYP17 activity thus decreases circulating levels of active androgens.

Abiraterone acetate is a steroidal androgen synthesis inhibitor which blocks the CYP17A1 enzyme. It is used in combination with prednisone for treatment of metastatic CRPC, before and after chemotherapy treatment. It is a prodrug of the active metabolite abiraterone. It also has some AR antagonist activity [E. Grist, R. Attard: The development of abiraterone acetate for castration-resistant prostate cancer. Urol. Ocol. 33, 289-294, 2015].

Seviteronel (developmental code VT-464; also known as (1S)-1-[6,7-bis(difluoromethoxy)naphthalen-2-yl]-2-methyl-1-(2H-triazol-4-yl)propan-1-ol) is a non-steroidal CYP17A1 inhibitor and in clinical studies for prostate cancer. It also has some AR antagonist activity [I. M. Bird, D. H. Abbott: The hunt for a selective 17,20 lyase inhibitor; learning lessons from nature. J. Steroid Biochem. Mol. Biol. 163, 136-146, 2016].

Galeterone (TOK-001 or VN/124-1, also known as (3S,8R,9S,10R,13S,14S)-17-(benzimidazol-1-yl)-10,13-dimethyl-2,3,4,7,8,9,11,12,14,15-decahydro-1H-cyclopenta[a]phenanthren-3-ol) is a steroidal antiandrogen under clinical development for the treatment of prostate cancer. It possesses a dual mechanism of action, acting as both an AR antagonist and a CYP17A1 inhibitor activity [I. M. Bird, D. H. Abbott: The hunt for a selective 17,20 lyase inhibitor; learning lessons from nature. J. Steroid Biochem. Mol. Biol. 163, 136-146, 2016].

Orteronel (TAK-700, also known as 6-(7-Hydroxy-6,7-dihydro-5H-pyrrolo[1,2-c]imidazol-7-yl)-N-methylnaphthalene-2-carboxamide) is a non-steroidal CYP17A1 inhibitor which completed clinical trials for metastatic CRPC treatment activity [I. M. Bird, D. H. Abbott: The hunt for a selective 17,20 lyase inhibitor; learning lessons from nature. J. Steroid Biochem. Mol. Biol. 163, 136-146, 2016].

Ketoconazole has antiandrogenic activity through at least two mechanisms of action [T. A. Yap, C. P. Carden et al.: Targeting CYP17: established and novel approaches in prostate cancer. Curr. Opin. Pharmacol. 8, 449-457, 2008]. It blocks both testicular and adrenal androgen biosynthesis by inhibiting the 17α-hydroxylase and 17,20-lyase, thus leading to a reduction in circulating testosterone levels. Due to its efficacy at reducing systemic androgen levels, ketoconazole has been used with some success as a treatment for androgen-dependent prostate cancer. Secondly, ketoconazole is an AR antagonist, competing with androgens such as testosterone and dihydrotestosterone (DHT) for binding to the AR.

ODM-204 is a dual AR antagonist/CYP17A inhibitor currently evaluated in patients with metastatic CRPC (Peltola et al. Eur Urol Focus 2018, DOI: 10.1016/j.euf2018.08.022). Component B may be administered by the oral, intravenous, topical, local installations, intraperitoneal or nasal route.

Component B may be in the form of a pharmaceutical formulation which is ready for use to be administered simultaneously, concurrently, separately or sequentially with component A and optionally component C as further described infra. The components A and B and optionally C may be administered independently of one another by the oral, intravenous, topical, local installations, intraperitoneal or nasal route.

### Combination

In accordance with another aspect, the present invention provides combinations of at least two components, preferably two components, component A and component B,

component A being a TTC, particularly PSMA-TTC as described infra,
and component B being an antiandrogen, particularly an AR antagonist selected from cyproterone acetate, bicalutamide, flutamide, nilutamide, enzalutamide, apalutamide, darolutamide or keto-darolutamide, or an AR degrader such as ARV-110, or an AR N-terminal domain binder such as EPI-506, or an antisense oligonucleotide that reduces AR expression such as EZN-4176 or AZD-5312, or an androgen synthesis inhibitor such as abiraterone, particularly abiraterone acetate, seviteronel, galeterone, orteronel or ketoconazole, or a dual AR antagonist and androgen synthesis inhibitor such as ODM-204.

In accordance with another aspect, the present invention provides combinations of at least two components, preferably two components, component A and component B, component A being PSMA-TTC, a hydrate, a solvate, or a pharmaceutically acceptable salt thereof, and component B being an antiandrogen, particularly an AR antagonist selected from bicalutamide, enzalutamide, apalutamide, darolutamide or keto-darolutamide, or an androgen synthesis inhibitor such as abiraterone, particularly abiraterone acetate.

In accordance with another aspect, the present invention covers a combination of any component A mentioned herein with any component B mentioned herein, optionally with any component C mentioned herein.

The combinations comprising at least two components A and B, preferably two components, as described and defined herein, are also referred to as “combinations of the present invention”.

The surprising behavior of a combination of the present invention is demonstrated herein with PSMA-TTC (“Compound A”) specifically disclosed in the Examples section.

In addition, a combination of the present invention comprising Compound A and enzalutamide is a preferred aspect of the invention.

Further, a combination of the present invention comprising Compound A and darolutamide (ODM-201) is another preferred aspect of the invention.

Further, the present invention covers a kit comprising:

component A: PSMA-TTC, a hydrate, a solvate, or a pharmaceutically acceptable salt thereof;
component B: an antiandrogen, or combinations of antiandrogens, as described supra.

In the kit optionally either or both of said components A and B in any of the above-mentioned combinations are in the form of a pharmaceutical composition which is ready for use to be administered simultaneously, concurrently, separately or sequentially. The components A and B may be administered independently of one another by the oral, intravenous, topical, local installations, intraperitoneal or nasal route. Preferably components A and B are administered by the oral route.

Further, the present invention covers a kit comprising:
component A: PSMA-TTC, a hydrate, a solvate, or a pharmaceutically acceptable salt thereof;
component B: an antiandrogen, or combinations of antiandrogens, as described supra; and, optionally,
component C: one or more, preferably one, further pharmaceutical agent(s),
   in which optionally either or all of said components A, B and C in any of the above-mentioned combinations are in the form of a pharmaceutical composition which is ready for use to be administered simultaneously, concurrently, separately or sequentially. The components A and B, optionally C, may be administered independently of one another by the oral, intravenous, topical, local installations, intraperitoneal or nasal route.

The term “component C” being at least one pharmaceutical agent includes the effective compound itself as well as its pharmaceutically acceptable salts, solvates, hydrates or stereoisomers as well as any pharmaceutical composition comprising such effective compound or its pharmaceutically acceptable salts, solvates, hydrates or stereoisomers. A list of such pharmaceutical agents of component C is being provided further below.

The combinations of component A and component B of this invention can be administered as the sole pharmaceutical agent or in combination with one or more further pharmaceutical agents C where the resulting combination of components A, B and C causes no unacceptable adverse effects. For example, the combinations of components A and B of this invention can be combined with component C, i.e. one or more further pharmaceutical agents, such as known anti-angiogenesis, anti-hyper-proliferative, antiinflammatory, analgesic, immunoregulatory, diuretic, antiarrhytmic, anti-hypercholsterolemia, anti-dyslipidemia, anti-diabetic or antiviral agents, and the like, as well as with admixtures and combinations thereof.

Optional pharmaceutical agents which can be added as component C to the combination of components A and B can be one or more pharmaceutical agents such as 131I-chTNT, abarelix, abiraterone, aclarubicin, ado-trastuzumab emtansine, afatinib, aflibercept, aldesleukin, alectinib, alemtuzumab, alendronic acid, alitretinoin, altretamine, amifostine, aminoglutethimide, hexyl aminolevulinate, amrubicin, amsacrine, anastrozole, ancestim, anethole dithiolethione, anetumab ravtansine, angiotensin II, antithrombin III, aprepitant, arcitumomab, arglabin, arsenic trioxide, asparaginase, axitinib, azacitidine, basiliximab, belotecan, bendamustine, besilesomab, belinostat, bevacizumab, bexarotene, bicalutamide, bisantrene, bleomycin, blinatumomab, bortezomib, buserelin, bosutinib, brentuximab vedotin, busulfan, cabazitaxel, cabozantinib, calcitonine, calcium folinate, calcium levofolinate, capecitabine, capromab, carboplatin, carboquone, carfilzomib, carmofur, carmustine, catumaxomab, CCS1477, celecoxib, celmoleukin, ceritinib, cetuximab, chlorambucil, chlormadinone, chlormethine, cidofovir, cinacalcet, cisplatin, cladribine, clodronic acid, clofarabine, cobimetinib, copanlisib, crisantaspase, crizotinib, cyclophosphamide, cyproterone, cytarabine, dacarbazine, dactinomycin, daratumumab, darbepoetin alfa, dabrafenib, dasatinib, daunorubicin, decitabine, degarelix, denileukin diftitox, denosumab, depreotide, deslorelin, dianhydrogalactitol, dexrazoxane, dibrospidium chloride, dianhydrogalactitol, diclofenac, dinutuximab, docetaxel, dolasetron, doxifluridine, doxorubicin, doxorubicin+estrone, dronabinol, eculizumab, edrecolomab, elliptinium acetate, elotuzumab, eltrombopag, endostatin, enocitabine, enzalutamide, epirubicin, epitiostanol, epoetin alfa, epoetin beta, epoetin zeta, eptaplatin, eribulin, erlotinib, esomeprazole, estradiol, estramustine, ethinylestradiol, etoposide, everolimus, exemestane, fadrozole, fentanyl, filgrastim, fluoxymesterone, floxuridine, fludarabine, fluorouracil, flutamide, folinic acid, formestane, fosaprepitant, fotemustine, fulvestrant, gadobutrol, gadoteridol, gadoteric acid meglumine, gadoversetamide, gadoxetic acid, gallium nitrate, ganirelix, gefitinib, gemcitabine, gemtuzumab, Glucarpidase, glutoxim, GM-CSF, goserelin, granisetron, granulocyte colony stimulating factor, GSK525762, histamine dihydrochloride, histrelin, hydroxycarbamide, I-125 seeds, lansoprazole, ibandronic acid, ibritumomab tiuxetan, ibrutinib, idarubicin, ifosfamide, imatinib, imiquimod, improsulfan, indisetron, incadronic acid, ingenol mebutate, interferon alfa, interferon beta, interferon gamma, iobitridol, iobenguane (123I), iomeprol, ipilimumab, irinotecan, Itraconazole, ixabepilone, ixazomib, lanreotide, lansoprazole, lapatinib, Iasocholine, lenalidomide, lenvatinib, lenograstim, lentinan, letrozole, leuprorelin, levamisole, levonorgestrel, levothyroxine sodium, lisuride, lobaplatin, lomustine, lonidamine, masoprocol, medroxyprogesterone, megestrol, melarsoprol, melphalan, mepitiostane, mercaptopurine, mesna, methadone, methotrexate, methoxsalen, methylaminolevulinate, methylprednisolone, methyltestosterone, metirosine, mifamurtide, miltefosine, miriplatin, mitobronitol, mitoguazone, mitolactol, mitomycin, mitotane, mitoxantrone, MK-8628, mogamulizumab, molgramostim, mopidamol, morphine hydrochloride, morphine sulfate, nabilone, nabiximols, nafarelin, naloxone+pentazocine, naltrexone, nartograstim, necitumumab, nedaplatin, nelarabine, neridronic acid, netupitant/palonosetron, nivolumabpentetreotide, nilotinib, nilutamide, nimorazole, nimotuzumab, nimustine, nintedanib, nitracrine, nivolumab, obinutuzumab, octreotide, ofatumumab, olaparib, omacetaxine mepesuccinate, omeprazole, ondansetron, oprelvekin, orgotein, orilotimod, osimertinib, oxaliplatin, oxycodone, oxymetholone, ozogamicine, p53 gene therapy, paclitaxel, palbociclib, palifermin, palladium-103 seed, palonosetron, pamidronic acid, panitumumab, panobinostat, pantoprazole, pazopanib, pegaspargase, PEG-epoetin beta (methoxy PEG-epoetin beta), pembrolizumab, pegfilgrastim, peginterferon alfa-2b, pemetrexed, pentazocine, pentostatin, peplomycin, Perflubutane, perfosfamide, Pertuzumab, picibanil, pilocarpine, pirarubicin, pixantrone, plerixafor, plicamycin, poliglusam, polyestradiol phosphate, polyvinylpyrrolidone+sodium hyaluronate, polysaccharide-K, pomalidomide, ponatinib, porfimer sodium, pralatrexate, prednimustine, prednisone, procarbazine, procodazole, propranolol, quinagolide, rabeprazole, racotumomab, radium-221, radotinib, raloxifene, raltitrexed, ramosetron, ramucirumab, ranimustine, rasburicase, razoxane, refametinib, regorafenib, risedronic acid, rhenium-186 etidronate, rituximab, rolapitant, romidepsin, romiplostim, romurtide, roniciclib, samarium (153Sm) lexidronam, sargramostim, satumomab, secretin, siltuximab, sipuleucel-T, sizofiran, sobuzoxane, sodium glycididazole, sonidegib, sorafenib, stanozolol, streptozocin, sunitinib, talaporfin, talimogene laherparepvec, tamibarotene, tamoxifen, tapentadol, tasonermin, teceleukin, technetium (99mTc) nofetumomab merpentan, 99mTc-HYNIC-[Tyr3]-octreotide, tegafur, tegafur+gimeracil+oteracil, temoporfin, temozolomide, temsirolimus, teniposide, testosterone, tetrofosmin, thalidomide, thiotepa, thymalfasin, thyrotropin alfa, tioguanine, tocilizumab, topotecan, toremifene, tositumomab, trabectedin, trametinib, tramadol, trastuzumab, trastuzumab emtansine, treosulfan, tretinoin, trifluridine+tipiracil, trilostane, triptorelin, trametinib, trofosfamide, thrombopoietin, tryptophan, ubenimex, valatinib, valrubicin, vandetanib, vapreotide, vemurafenib, vinblastine, vincristine, vindesine, vinflunine, vinorelbine, vismodegib, vorinostat, vorozole, yttrium-90 glass microspheres, ZEN003694, zinostatin, zinostatin stimalamer, zoledronic acid, zorubicin or combinations thereof.

Generally, the use of pharmaceutical agents as component C in combination with a combination of components A and B of the present invention will serve to:
(1) yield better efficacy in reducing the growth of a tumor and/or metastasis or even eliminate the tumor and/or metastasis as compared to administration of either agent alone,
(2) provide for the administration of lesser amounts of the administered chemotherapeutic agents,
(3) provide for a chemotherapeutic treatment that is well tolerated in the patient with fewer deleterious pharmacological complications than observed with single agent chemotherapies and certain other combined therapies,
(4) provide for treating a broader spectrum of different cancer types in mammals, especially humans,
(5) provide for a higher response rate among treated patients,
(6) provide for a longer survival time among treated patients compared to standard chemotherapy treatments,
(7) provide a longer time for tumor progression, and/or
(8) yield efficacy and tolerability results at least as good as those of the agents used alone, compared to known instances where other cancer agent combinations produce antagonistic effects.

Further, the present invention covers a pharmaceutical composition comprising a combination of the present invention as described herein together with one or more pharmaceutically acceptable excipients.

Further, the present invention covers a pharmaceutical composition comprising a combination of at least two components, particularly of two components, component A and component B, component A being PSMA-TTC a hydrate, a solvate, or a pharmaceutically acceptable salt thereof, and component B being an antiandrogen as described supra, particularly an antiandrogen selected from the AR antagonists cyproterone acetate, bicalutamide, flutamide, nilutamide, enzalutamide, apalutamide, darolutamide or keto-darolutamide, or an AR degrader such as ARV-110, or an AR N-terminal domain binder such as EPI-506, or an antisense oligonucleotide that reduces AR expression such as EZN-4176 or AZD-5312, or an androgen synthesis inhibitor such as abiraterone, particularly abiraterone acetate, seviteronel, galeterone, orteronel or ketoconazole, or a dual AR antagonist and CYP17A inhibitor such as ODM-204.

together with one or more pharmaceutically acceptable excipients.

Further, the present invention covers a pharmaceutical composition comprising a combination of at least two components, particularly of two components, component A and component B, component A being an inhibitor of ATR kinase as described supra, particularly Compound A or a stereoisomer, a tautomer, an N-oxide, a hydrate, a solvate, or a pharmaceutically acceptable salt thereof, and component B being an antiandrogen as described supra; optionally with any component C mentioned herein, together with one or more pharmaceutically acceptable excipients.

A preferred aspect of the present invention covers a pharmaceutical composition comprising a combination of at least two components, particularly of two components, component A and component B, component A being Compound A or a stereoisomer, a tautomer, an N-oxide, a hydrate, a solvate, or a pharmaceutically acceptable salt thereof, and component B being darolutamide (=ODM-201) or enzalutamide, together with one or more pharmaceutically acceptable excipients.

In another embodiment the components A and B, and optionally component C, are present in separate formulations.

In another embodiment the components A and B, and optionally component C, are present in a joint formulation.

Pharmaceutically acceptable excipients are non-toxic, preferably they are non-toxic and inert.

Pharmaceutically acceptable excipients include, inter alia,
fillers and excipients (for example cellulose, microcrystalline cellulose, such as, for example, Avicel®, lactose, mannitol, starch, calcium phosphate such as, for example, Di-Cafos®),
ointment bases (for example petroleum jelly, paraffins, triglycerides, waxes, wool wax, wool wax alcohols, lanolin, hydrophilic ointment, polyethylene glycols),
bases for suppositories (for example polyethylene glycols, cacao butter, hard fat)
solvents (for example water, ethanol, Isopropanol, glycerol, propylene glycol, medium chain-length triglycerides fatty oils, liquid polyethylene glycols, paraffins),
surfactants, emulsifiers, dispersants or wetters (for example sodium dodecyle sulphate, lecithin, phospholipids, fatty alcohols such as, for example, Lanette®, sorbitan fatty acid esters such as, for example, Span®, polyoxyethylene sorbitan fatty acid esters such as, for example, Tween®, polyoxyethylene fatty acid glycerides such as, for example, Cremophor®, polyoxethylene fatty acid esters, polyoxyethylene fatty alcohol ethers, glycerol fatty acid esters, poloxamers such as, for example, Pluronic®),
buffers and also acids and bases (for example phosphates, carbonates, citric acid, acetic acid, hydrochloric acid, sodium hydroxide solution, ammonium carbonate, trometamol, triethanolamine)
isotonicity agents (for example glucose, sodium chloride),
adsorbents (for example highly-disperse silicas)
viscosity-increasing agents, gel formers, thickeners and/or binders (for example polyvinylpyrrolidon, methylcellulose, hydroxypropylmethylcellulose, hydroxypropylcellulose, carboxymethylcellulose-sodium, starch, carbomers, polyacrylic acids such as, for example, Carbopol®, alginates, gelatine),
disintegrants (for example modified starch, carboxymethylcellulose-sodium, sodium starch glycolate such as, for example, Explotab®, cross-linked polyvinylpyrrolidon, croscarmellose-sodium such as, for example, AcDiSol®),
flow regulators, lubricants, glidant and mould release agents (for example magnesium stearate, stearic acid, talc, highly-disperse silicas such as, for example, Aerosil®),
coating materials (for example sugar, shellac) and film formers for films or diffusion membranes which dissolve rapidly or in a modified manner (for example polyvinylpyrrolidones such as, for example, Kollidon®, polyvinyl alcohol, hydroxypropylmethylcellulose, hydroxypropylcellulose, ethylcellulose, hydroxypropylmethylcellulose phthalate, cellulose acetate, cellulose acetate phthalate, polyacrylates, polymethacrylates such as, for example, Eudragit®),
capsule materials (for example gelatine, hydroxypropylmethylcellulose),
synthetic polymers (for example polylactides, polyglycolides, polyacrylates, polymethacrylates such as, for example, Eudragit®, polyvinylpyrrolidones such as, for example, Kollidon®, polyvinyl alcohols, polyvinyl acetates, polyethylene oxides, polyethylene glycols and their copolymers and blockcopolymers),
plasticizers (for example polyethylene glycols, propylene glycol, glycerol, triacetine, triacetyl citrate, dibutyl phthalate),
penetration enhancers,
stabilisers (for example antioxidants such as, for example, ascorbic acid, ascorbyl palmitate, sodium ascorbate, butylhydroxyanisole, butylhydroxytoluene, propyl gallate),
preservatives (for example parabens, sorbic acid, thiomersal, benzalkonium chloride, chlorhexidine acetate, sodium benzoate),
colourants (for example inorganic pigments such as, for example, iron oxides, titanium dioxide),
flavourings, sweeteners, flavour- and/or odour-masking agents.

Further excipients and procedures are described in the following references, each of which is incorporated herein by reference: Powell, M. F. et al., “Compendium of Excipients for Parenteral Formulations” PDA Journal of Pharmaceutical Science & Technology 1998, 52(5), 238-311; Strickley, R. G “Parenteral Formulations of Small Molecule Therapeutics Marketed in the United States (1999)-Part-1” PDA Journal of Pharmaceutical Science & Technology 1999, 53(6), 324-349; and Nema, S. et al., “Excipients and Their Use in Injectable Products” PDA Journal of Pharmaceutical Science & Technology 1997, 51(4), 166-171.

The components A, B and C may be administered independently of one another by the oral, intravenous, topical, local installations, intraperitoneal or nasal route.

Components A, B and C are preferably administered orally.

The pharmaceutical composition (formulation) varies by the route of administration. Components of this invention can be tableted with conventional tablet bases such as lactose, sucrose and cornstarch in combination with binders such as acacia, corn starch or gelatin, disintegrating agents intended to assist the break-up and dissolution of the tablet following administration such as potato starch, alginic acid, corn starch, and guar gum, gum tragacanth, acacia, lubricants intended to improve the flow of tablet granulation and to prevent the adhesion of tablet material to the surfaces of the tablet dies and punches, for example talc, stearic acid, or magnesium, calcium or zinc stearate, dyes, coloring agents, and flavoring agents such as peppermint, oil of wintergreen, or cherry flavoring, intended to enhance the aesthetic qualities of the tablets and make them more acceptable to the patient. Suitable excipients for use in oral liquid dosage forms include dicalcium phosphate and diluents such as water and alcohols, for example, ethanol, benzyl alcohol, and polyethylene alcohols, either with or without the addition of a pharmaceutically acceptable surfactant, suspending agent or emulsifying agent. Various other materials may be present as coatings or to otherwise modify the physical form of the dosage unit. For instance tablets, pills or capsules may be coated with shellac, sugar or both.

Dispersible powders and granules are suitable for the preparation of an aqueous suspension. They provide the active ingredient in admixture with a dispersing or wetting agent, a suspending agent and one or more preservatives. Suitable dispersing or wetting agents and suspending agents are exemplified by those already mentioned above. Additional excipients, for example those sweetening, flavoring and coloring agents described above, may also be present.

Components of this invention can also be in the form of oil-in-water emulsions. The oily phase may be a vegetable oil such as liquid paraffin or a mixture of vegetable oils. Suitable emulsifying agents may be (1) naturally occurring gums such as gum acacia and gum tragacanth, (2) naturally occurring phosphatides such as soy bean and lecithin, (3) esters or partial esters derived from fatty acids and hexitol anhydrides, for example, sorbitan monooleate, (4) condensation products of said partial esters with ethylene oxide, for example, polyoxyethylene sorbitan monooleate. The emulsions may also contain sweetening and flavoring agents.

Oily suspensions can be formulated by suspending the active ingredient in a vegetable oil such as, for example, arachis oil, olive oil, sesame oil or coconut oil, or in a mineral oil such as liquid paraffin. The oily suspensions may contain a thickening agent such as, for example, beeswax, hard paraffin, or cetyl alcohol. The suspensions may also contain one or more preservatives, for example, ethyl or n-propyl p-hydroxybenzoate; one or more coloring agents; one or more flavoring agents; and one or more sweetening agents such as sucrose or saccharin.

Syrups and elixirs can be formulated with sweetening agents such as, for example, glycerol, propylene glycol, sorbitol or sucrose. Such formulations may also contain a demulcent, and preservative, such as methyl and propyl parabens and flavoring and coloring agents.

Components of this invention can also be administered parenterally, that is, subcutaneously, intravenously, intraocularly, intrasynovially, intramuscularly, or interperitoneally, as injectable dosages of the compound in preferably a pharmaceutically acceptable diluent with a pharmaceutical carrier which can be a sterile liquid or mixture of liquids such as water, saline, aqueous dextrose and related sugar solutions, an alcohol such as ethanol, isopropanol, or hexadecyl alcohol, glycols such as propylene glycol or polyethylene glycol, glycerol ketals such as 2,2-dimethyl-1,1-dioxolane-4-methanol, ethers such as poly(ethylene glycol) 400, an oil, a fatty acid, a fatty acid ester or, a fatty acid glyceride, or an acetylated fatty acid glyceride, with or without the addition of a pharmaceutically acceptable surfactant such as a soap or a detergent, suspending agent such as pectin, carbomers, methycellulose, hydroxypropylmethylcellulose, or carboxymethylcellulose, or emulsifying agent and other pharmaceutical adjuvants.

Illustrative of oils which can be used in the parenteral formulations of this invention are those of petroleum, animal, vegetable, or synthetic origin, for example, peanut oil, soybean oil, sesame oil, cottonseed oil, corn oil, olive oil, petrolatum and mineral oil. Suitable fatty acids include oleic acid, stearic acid, isostearic acid and myristic acid. Suitable fatty acid esters are, for example, ethyl oleate and isopropyl myristate. Suitable soaps include fatty acid alkali metal, ammonium, and triethanolamine salts and suitable detergents include cationic detergents, for example dimethyl dialkyl ammonium halides, alkyl pyridinium halides, and alkylamine acetates; anionic detergents, for example, alkyl, aryl, and olefin sulfonates, alkyl, olefin, ether, and monoglyceride sulfates, and sulfosuccinates; non-ionic detergents, for example, fatty amine oxides, fatty acid alkanolamides, and poly(oxyethylene-oxypropylene)s or ethylene oxide or propylene oxide copolymers; and amphoteric detergents, for example, alkyl-beta-aminopropionates, and 2-alkylimidazoline quarternary ammonium salts, as well as mixtures.

The parenteral compositions of this invention will typically contain from about 0.5% to about 25% by weight of the active ingredient in solution. Preservatives and buffers may also be used advantageously. In order to minimize or eliminate irritation at the site of injection, such compositions may contain a non-ionic surfactant having a hydrophile-lipophile balance (HLB) preferably of from about 12 to about 17. The quantity of surfactant in such formulation preferably ranges from about 5% to about 15% by weight. The surfactant can be a single component having the above HLB or can be a mixture of two or more components having the desired HLB.

Illustrative of surfactants used in parenteral formulations are the class of polyethylene sorbitan fatty acid esters, for example, sorbitan monooleate and the high molecular weight adducts of ethylene oxide with a hydrophobic base, formed by the condensation of propylene oxide with propylene glycol.

The pharmaceutical compositions of the present invention can be in the form of sterile injectable aqueous suspensions. Such suspensions may be formulated according to known methods using suitable dispersing or wetting agents and suspending agents such as, for example, sodium carboxymethylcellulose, methylcellulose, hydroxypropylmethyl-cellulose, sodium alginate, polyvinylpyrrolidone, gum tragacanth and gum acacia; dispersing or wetting agents which may be a naturally occurring phosphatide such as lecithin, a condensation product of an alkylene oxide with a fatty acid, for example, polyoxyethylene stearate, a condensation product of ethylene oxide with a long chain aliphatic alcohol, for example, heptadeca-ethyleneoxycetanol, a condensation product of ethylene oxide with a partial ester derived form a fatty acid and a hexitol such as polyoxyethylene sorbitol monooleate, or a condensation product of an ethylene oxide with a partial ester derived from a fatty acid and a hexitol anhydride, for example polyoxyethylene sorbitan monooleate.

The sterile injectable preparation can also be a sterile injectable solution or suspension in a non-toxic parenterally acceptable diluent or solvent. Diluents and solvents that may be employed are, for example, water, Ringer's solution, isotonic sodium chloride solutions and isotonic glucose solutions. In addition, sterile fixed oils are conventionally employed as solvents or suspending media. For this purpose, any bland, fixed oil may be employed including synthetic mono- or diglycerides. In addition, fatty acids such as oleic acid can be used in the preparation of injectables.

Components of the invention can also be administered in the form of suppositories for rectal administration of the drug. These components can be prepared by mixing the drug with a suitable non-irritation excipient which is solid at ordinary temperatures but liquid at the rectal temperature and will therefore melt in the rectum to release the drug. Such materials are, for example, cocoa butter and polyethylene glycol.

Another formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art (see, e.g., U.S. Pat. No. 5,023,252, issued Jun. 11, 1991, incorporated herein by reference). Such patches may be constructed for continuous, pulsatile, or on demand delivery of pharmaceutical agents.

Controlled release formulations for parenteral administration include liposomal, polymeric microsphere and polymeric gel formulations that are known in the art.

It can be desirable or necessary to introduce a component of the present invention to the patient via a mechanical delivery device. The construction and use of mechanical delivery devices for the delivery of pharmaceutical agents is well known in the art. Direct techniques for, for example, administering a drug directly to the brain usually involve placement of a drug delivery catheter into the patient's ventricular system to bypass the blood-brain barrier. One such implantable delivery system, used for the transport of agents to specific anatomical regions of the body, is described in U.S. Pat. No. 5,011,472, issued Apr. 30, 1991.

In accordance with another aspect, the present invention concerns the use of the combination of the present invention as described supra for the treatment or prophylaxis of a disease, preferably a hyper-proliferative disease as described infra and/or metastases thereof, preferably metastases in bone.

In accordance with another aspect, the present invention concerns the combination of the present invention as described supra for use in the treatment or prophylaxis of a hyper-proliferative disease as described infra, particularly of prostate cancer, preferably in the treatment of castration-resistant prostate cancer (CRPC) or of metastatic hormone sensitive prostate cancer (mHSPC).

In accordance with another aspect, the present invention concerns the kit as described supra for the treatment or prophylaxis of a disease, preferably a hyper-proliferative disease as described infra.

In accordance with another aspect, the present invention concerns the kit as described supra for use in the treatment or prophylaxis of a hyper-proliferative disease as described infra, particularly of prostate cancer, preferably in the treatment of castration-resistant prostate cancer (CRPC) or of metastatic hormone sensitive prostate cancer (mHSPC).

In accordance with another aspect, the present invention concerns the pharmaceutical composition as described supra for the treatment or prophylaxis of a disease, preferably a hyper-proliferative disease as described infra.

In accordance with another aspect, the present invention concerns the pharmaceutical composition as described supra for use in the treatment or prophylaxis of a hyper-proliferative disease as described infra, particularly of prostate cancer, preferably in the treatment of castration-resistant prostate cancer (CRPC) or of metastatic hormone sensitive prostate cancer (mHSPC).

In accordance with another aspect, the present invention covers the use of such combinations as described supra for the preparation of a medicament for the treatment or prophylaxis of a disease, preferably a hyper-proliferative disease as described infra.

In accordance with another aspect, the present invention covers the use of such kit as described supra for the preparation of a medicament for the treatment or prophylaxis of a disease, preferably a hyper-proliferative disease as described infra.

In accordance with another aspect, the present invention covers the use of such pharmaceutical composition as described supra for the preparation of a medicament for the treatment or prophylaxis of a disease, preferably a hyper-proliferative disease as described infra.

In accordance with another aspect, the present invention concerns methods for the treatment and/or prophylaxis of a disease, preferably a hyper-proliferative disease as described infra using an effective amount of the combination of the present invention as described supra.

In accordance with another aspect, the present invention concerns methods for the treatment and/or prophylaxis of a disease, preferably a hyper-proliferative disease as described infra using an effective amount of the kit or pharmaceutical composition as described supra.

In accordance with another aspect, the present invention concerns a method of treating a disease in a patient, preferably a hyper-proliferative disease as described infra comprising
a) administering component A being PSMA-TTC, a hydrate, a solvate, or a pharmaceutically acceptable salt thereof, and
b) administering component B being an antiandrogen as described supra, particularly an antiandrogen selected from AR antagonists such as cyproterone acetate, bicalutamide, flutamide, nilutamide, enzalutamide, apalutamide, darolutamide or keto-darolutamide, or an AR degrader such as ARV-110, or an AR N-terminal domain binder such as EPI-506, or an antisense oligonucleotide that reduces AR expression such as EZN-4176 or AZD-5312, or an androgen synthesis inhibitor such as abiraterone, particularly abiraterone acetate, seviteronel, galeterone, orteronel or ketoconazole, or a dual AR antagonist and androgen synthesis inhibitor such as ODM-204.

In accordance with another aspect, the present invention concerns a method of treating a disease in a patient, preferably a hyper-proliferative disease as described infra comprising
a) administering component A being PSMA-TTC, a hydrate, a solvate, or a pharmaceutically acceptable salt thereof, and
b) administering component B being an antiandrogen as described supra, particularly an antiandrogen selected from AR antagonists such as cyproterone acetate, bicalutamide, flutamide, nilutamide, enzalutamide, apalutamide, darolutamide or keto-darolutamide, or an AR degrader such as ARV-110, or an AR N-terminal domain binder such as EPI-506, or an antisense oligonucleotide that reduces AR expression such as EZN-4176 or AZD-5312, or an androgen synthesis inhibitor such as abiraterone, particularly abiraterone acetate, seviteronel, galeterone, orteronel or ketoconazole, or a dual AR antagonist and androgen synthesis inhibitor such as ODM-204.

In accordance with another aspect, the present invention concerns a method of treating a hyper-proliferative disease as described infra, particularly of treating prostate cancer, preferably of treating castration-resistant prostate cancer (CRPC) or of metastatic hormone sensitive prostate cancer (mHSPC), comprising
a) administering Compound A, a hydrate, a solvate, or a pharmaceutically acceptable salt thereof, and
b) administering component B being an antiandrogen selected from enzalutamide and darolutamide (ODM-201).

In accordance with another aspect, the present invention concerns a method of treating a hyper-proliferative disease as described infra, particularly of treating prostate cancer, preferably of treating castration-resistant prostate cancer (CRPC) or of metastatic hormone sensitive prostate cancer (mHSPC), comprising
a) administering Compound A, a hydrate, a solvate, or a pharmaceutically acceptable salt thereof, and
b) administering component B being an antiandrogen selected from enzalutamide and darolutamide (ODM-201), wherein Compound A and component B are administered concurrently.

In accordance with another aspect, the present invention concerns a method of treating a hyper-proliferative disease as described infra, particularly of treating prostate cancer, preferably of treating castration-resistant prostate cancer (CRPC) or of metastatic hormone sensitive prostate cancer (mHSPC), comprising
a) administering Compound A, a hydrate, a solvate, or a pharmaceutically acceptable salt thereof, and
b) administering component B being an antiandrogen selected from enzalutamide and darolutamide (ODM-201), wherein Compound A is administered prior to component B.

In accordance with another aspect, the present invention concerns a method of treating a hyper-proliferative disease as described infra, particularly of treating breast cancer or prostate cancer, preferably of treating castration-resistant prostate cancer (CRPC) or of metastatic hormone sensitive prostate cancer (mHSPC), comprising
a) administering Compound A, a hydrate, a solvate, or a pharmaceutically acceptable salt thereof, and
b) administering component B being an antiandrogen selected from enzalutamide and darolutamide (ODM-201), wherein component B is administered prior to Compound A.

In accordance with another aspect, the present invention concerns a method of treating a disease in a patient, preferably a hyper-proliferative disease as described infra comprising
a) administering, Compound A, a hydrate, a solvate, or a pharmaceutically acceptable salt thereof, and
b) administering component B being an antiandrogen as described supra, particularly an antiandrogen selected from AR antagonists such as cyproterone acetate, bicalutamide, flutamide, nilutamide, enzalutamide, apalutamide, darolutamide or keto-darolutamide, or an AR degrader such as ARV-110, or an AR N-terminal domain binder such as EPI-506, or an antisense oligonucleotide that reduces AR expression such as EZN-4176 or AZD-5312, or an androgen synthesis inhibitor such as abiraterone, particularly abiraterone acetate, seviteronel, galeterone, orteronel or ketoconazole, or a dual AR antagonist and androgen synthesis inhibitor such as ODM-204.
c) administering component C being a pharmaceutical agent as described supra.

The combinations, kits or pharmaceutical compositions of the present invention thus can be used for the treatment or prophylaxis of hyper-proliferative diseases, including diseases of uncontrolled cell growth, proliferation and/or survival, inappropriate cellular immune responses, or inappropriate cellular inflammatory responses, or diseases which are accompanied with uncontrolled cell growth, proliferation and/or survival, inappropriate cellular immune responses, or inappropriate cellular inflammatory responses, particularly in which the uncontrolled cell growth, proliferation and/or survival, inappropriate cellular immune responses, or inappropriate cellular inflammatory responses, such as, for example, haematological tumors and/or metastases thereof, solid tumors, and/or metastases thereof, e.g. leukemias, multiple myeloma thereof and myelodysplastic syndrome, malignant lymphomas, breast tumors including and bone metastases thereof, tumors of the thorax including non-small cell and small cell lung tumors and bone metastases thereof, gastrointestinal tumors, endocrine tumors, mammary and other gynaecological tumors and bone metastases thereof, urological tumors including renal, bladder and prostate tumors, skin tumors, and sarcomas, and/or metastases thereof.

The term “inappropriate” within the context of the present invention, in particular in the context of “inappropriate cellular immune responses, or inappropriate cellular inflammatory responses”, as used herein, is to be understood as preferably meaning a response which is less than, or greater than normal, and which is associated with, responsible for, or results in, the pathology of said diseases.

Combinations, kits or pharmaceutical compositions of the present invention might be utilized to inhibit, block, reduce, decrease, etc., cell proliferation and/or cell division, and/or produce apoptosis.

This invention includes a method comprising administering to a mammal in need thereof, including a human, an amount of a component A and an amount of component B of this invention, or a pharmaceutically acceptable salt, isomer, polymorph, metabolite, hydrate, solvate or ester thereof, which is effective to treat the hyper-proliferative disease.

Hyper-proliferative diseases include but are not limited, e.g., psoriasis, keloids, and other hyperplasias affecting the skin, benign prostate hyperplasia (BPH), as well as malignant neoplasia. Examples of malignant neoplasia treatable with the compounds according to the present invention include solid and hematological tumors. Solid tumors can be exemplified by tumors of the breast, bladder, bone, brain, central and peripheral nervous system, colon, anum, endocrine glands (e.g. thyroid and adrenal cortex), esophagus, endometrium, germ cells, head and neck, kidney, liver, lung, larynx and hypopharynx, mesothelioma, ovary, pancreas, prostate, rectum, renal, small intestine, soft tissue, testis, stomach, skin, ureter, vagina and vulva. Malignant neoplasias include inherited cancers exemplified by Retinoblastoma and Wilms tumor. In addition, malignant neoplasias include primary tumors in said organs and corresponding secondary tumors in distant organs (“tumor metastases”). Hematological tumors can be exemplified by aggressive and indolent forms of leukemia and lymphoma, namely non-Hodgkins disease, chronic and acute myeloid leukemia (CML/AML), acute lymphoblastic leukemia (ALL), Hodgkins disease, multiple myeloma and T-cell lymphoma. Also included are myelodysplastic syndrome, plasma cell neoplasia, paraneoplastic syndromes, and cancers of unknown primary site as well as AIDS related malignancies.

Examples of breast cancer include, but are not limited to invasive ductal carcinoma, invasive lobular carcinoma, ductal carcinoma in situ, and lobular carcinoma in situ, particularly with bone metastases.

Examples of cancers of the respiratory tract include, but are not limited to small-cell and non-small-cell lung carcinoma, as well as bronchial adenoma and pleuropulmonary blastoma.

Examples of brain cancers include, but are not limited to brain stem and hypophtalmic glioma, cerebellar and cerebral astrocytoma, medulloblastoma, ependymoma, as well as neuroectodermal and pineal tumor.

Tumors of the male reproductive organs include, but are not limited to prostate and testicular cancer. Tumors of the female reproductive organs include, but are not limited to endometrial, cervical, ovarian, vaginal, and vulvar cancer, as well as sarcoma of the uterus.

Tumors of the digestive tract include, but are not limited to anal, colon, colorectal, esophageal, gallbladder, gastric, pancreatic, rectal, small-intestine, and salivary gland cancers.

Tumors of the urinary tract include, but are not limited to bladder, penile, kidney, renal pelvis, ureter, urethral and human papillary renal cancers.

Eye cancers include, but are not limited to intraocular melanoma and retinoblastoma.

Examples of liver cancers include, but are not limited to hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to squamous cell carcinoma, Kaposi's sarcoma, malignant melanoma, Merkel cell skin cancer, and non-melanoma skin cancer.

Head-and-neck cancers include, but are not limited to laryngeal, hypopharyngeal, nasopharyngeal, oropharyngeal cancer, lip and oral cavity cancer and squamous cell. Lymphomas include, but are not limited to AIDS-related lymphoma, non-Hodgkin's lymphoma, cutaneous T-cell lymphoma, Burkitt lymphoma, Hodgkin's disease, and lymphoma of the central nervous system.

Sarcomas include, but are not limited to sarcoma of the soft tissue, osteosarcoma, malignant fibrous histiocytoma, lymphosarcoma, and rhabdomyosarcoma.

Leukemias include, but are not limited to acute myeloid leukemia, acute lymphoblastic leukemia, chronic lymphocytic leukemia, chronic myelogenous leukemia, and hairy cell leukemia.

These diseases have been well characterized in humans, but also exist with a similar etiology in other mammals, and can be treated by administering pharmaceutical compositions of the present invention.

Combinations of the present invention might also be used for treating diseases associated with excessive and/or abnormal angiogenesis.

Inappropriate and ectopic expression of angiogenesis can be deleterious to an organism. A number of pathological conditions are associated with the growth of extraneous blood vessels. These include, e.g., diabetic retinopathy, ischemic retinal-vein occlusion, and retinopathy of prematurity [Aiello et al. New Engl. J. Med. 1994, 331, 1480; Peer et al. Lab. Invest. 1995, 72, 638], age-related macular degeneration [AMD; see, Lopez et al. Invest. Opththalmol. Vis. Sci. 1996, 37, 855], neovascular glaucoma, psoriasis, retrolental fibroplasias, angiofibroma, inflammation, rheumatoid arthritis (RA), restenosis, in-stent restenosis, vascular graft restenosis, etc. In addition, the increased blood supply associated with cancerous and neoplastic tissue, encourages growth, leading to rapid tumor enlargement and metastases. Moreover, the growth of new blood and lymph vessels in a tumor provides an escape route for renegade cells, encouraging metastases and the consequence spread of the cancer. Thus, combinations of the present invention can be utilized to treat and/or prevent any of the aforementioned angiogenesis diseases, e.g., by inhibiting and/or reducing blood vessel formation; by inhibiting, blocking, reducing, decreasing, etc. endothelial cell proliferation or other types involved in angiogenesis, as well as causing cell death or apoptosis of such cell types.

The term “prostate cancer” as used herein means any histology type of prostate cancer including but not limited to acinar adenocarcinoma, ductal adenocarcinoma, transitional cell (or urothelial) cancer, squamous cell cancer, carcinoid, small cell cancer, sarcomas and sarcomatoid cancers, particularly acinar adenocarcinoma, metastatic hormone sensitive prostate cancer (mHSPC), castration resistant prostate cancer (CRPC), particularly stage M0 castration-resistant prostate cancer (M0 CRPC) or stage M1 castration-resistant prostate cancer (M1 CRPC).

The terms “M0” and “M1” (including M1a, M1b, M1c) are used in accordance with the “TNM staging system” for prostate cancer developed by the American Joint Committee on Cancer as further described in “TNM CLASSIFICATION OF MALIGNANT TUMORS”, 7th edition Edited by James D. Brierley, Mary K. Gospodarowicz, Christian Wittekind, Published by UICC 2011.

According to said TNM classification and as used herein the term “M0 CRPC” means that there are no distant metastases and that the CRPC has not spread to other parts of the body. The term “M1 CRPC” as used herein means that there are distant metastases and that the CRPC has spread to distant parts of the body.

In particular, the present invention covers the treatment of prostate cancer, particularly the treatment of metastatic hormone sensitive prostate cancer (mHSPC) or of castration-resistant prostate cancer (CRPC).

In another embodiment the combination/kit/pharmaceutical composition of the present invention are used in the treatment of prostate cancer or breast cancer, particularly in the treatment of mHSPC, M0 CRPC, M1 CRPC or breast cancer.

In another embodiment of the use of the combination/kit/pharmaceutical composition of the present invention, the castration resistant prostate cancer (CRPC) is stage M0 castration resistant prostate cancer (M0 CRPC) or stage M1 castration-resistant prostate cancer (M1 CRPC).

In another embodiment of the use of the combination/kit/pharmaceutical composition of the present invention the castration resistant prostate cancer (CRPC) is stage M0 castration resistant prostate cancer (M0 CRPC) or stage M1 castration-resistant prostate cancer (M1 CRPC), and the subject to be treated is chemotherapy-naïve.

The term “chemotherapy-naïve” as used herein means that the subject, prior to the treatment with the combination/kit/pharmaceutical composition of the present invention has not received a chemotherapy.

In another embodiment of the use of the combination/kit/pharmaceutical composition of the present invention the castration resistant prostate cancer (CRPC) is stage M0 castration resistant prostate cancer (M0 CRPC) or stage M1 castration-resistant prostate cancer (M1 CRPC), and the subject to be treated is a subject, wherein the subject has received a chemotherapy prior to the treatment with the combination/kit/pharmaceutical composition of the present invention.

The term “chemotherapy” as used herein means a category of cancer treatment that uses one or more chemotherapeutic agents as part of a standardized chemotherapy regimen. Chemotherapeutic agents are rather non-specific agents including but not limited to alkylating agents, anthracyclines, taxanes, epothilones, histone deacetylase inhibitors, inhibitors of topoisomerase I, inhibitors of topoisomerase II, nucleotide analogues, platinum-based agents, vinca alkaloids.

### Dose and Administration

Component A

Based upon standard laboratory techniques known to evaluate compounds useful for the treatment of hyper-proliferative diseases and angiogenic diseases, by standard toxicity tests and by standard pharmacological assays for the determination of treatment of the conditions identified above in mammals, and by comparison of these results with the results of known medicaments that are used to treat these conditions, the effective dosage of the compounds of this invention can readily be determined for treatment of each desired indication. The amount of the active ingredients to be administered in the treatment of one of these conditions can vary widely according to such considerations as the particular component and dosage unit employed, the mode of administration, the period of treatment, the age and sex of the patient treated, and the nature and extent of the condition treated.

A preferred dosage regimen for TTC injection is between 0.25 and 4 MBq per subject on day one of each cycle, at total antibody doses ranging between 20-100 mg. Each cycle preferably has a duration of 6 weeks (42 days) or up to 12 weeks (84 days). Each patient preferably undergoes up to 6 treatment cycles. Doses of total antibody may be adjusted by injecting the “cold” total antibody 1 hour prior to the administration of the radiolabelled compound. Doses up to 7.4 MBq per cycle per subject have been administered in a Phase I trial. The observed adverse reactions at this dose were reversible myelosuppression . . . .

TTCs are to be administered intravenously by a qualified personnel as slow bolus injection or infusion. An intravenous access line should be used for administration of a TTC.

The total amount of the active ingredients to be administered will generally range from about 0.001 mg/kg to about 200 mg/kg body weight per day, and preferably from about 0.01 mg/kg to about 50 mg/kg body weight per day. Clinically useful dosing schedules of a compound will range from one to three times a day dosing to once every four weeks dosing. In addition, “drug holidays” in which a patient is not dosed with a drug for a certain period of time, may be beneficial to the overall balance between pharmacological effect and tolerability. A unit dosage may contain from about 0.5 mg to about 1500 mg of active ingredient, and can be administered one or more times per day or less than once a day. The average daily dosage for administration by injection, including intravenous, intramuscular, subcutaneous and parenteral injections, and use of infusion techniques will preferably be from 0.01 to 200 mg/kg of total body weight.

Component B

Component B being an antiandrogen, particularly an AR antagonist selected from cyproterone acetate, bicalutamide, flutamide, nilutamide, enzalutamide, apalutamide, darolutamide or keto-darolutamide, or an AR degrader such as ARV-110, or an AR N-terminal domain binder such as EPI-506, or an androgen synthesis inhibitor such as abiraterone, particularly abiraterone acetate, seviteronel, galeterone, orteronel or ketoconazole, or a dual AR antagonist and androgen synthesis inhibitor such as ODM-204. can be administered to a patient at a dosage which can range from about 1 to about 2000 mg per day. Antisense oligonucleotide such as EZN-4176 or AZD-5312 can be administered to a patient at a dosage of weekly one-hour i.v. infusions, with or without drug holidays.

Also, the agents can be administered in conventional amounts routinely used in cancer chemotherapy. Typically, the following treatments are used: 100 mg two or three times a day (cyproterone acetate), 50 mg daily (bicalutamide), 250 mg three times a day (flutamide), 150 or 300 mg daily (nilutamide), 160 mg daily (enzalutamide), 240 mg daily (apalutamide), 1000 mg daily (abiraterone), 600 mg twice a day (darolutamide), 200 or 400 mg three times a day (ketoconazole), 300 mg twice daily (orteronel).

Of course the specific initial and continuing dosage regimen for each patient will vary according to the nature and severity of the condition as determined by the attending diagnostician, the activity of the specific compounds employed, the age and general condition of the patient, time of administration, route of administration, rate of excretion of the drug, drug combinations, and the like. The desired mode of treatment and number of doses of a compound of the present invention or a pharmaceutically acceptable salt or ester or composition thereof can be ascertained by those skilled in the art using conventional treatment tests.

Suitable dose(s), administration regime(s) and administration route(s) for component B being an antiandrogen selected from an AR antagonist such as cyproterone acetate, bicalutamide, flutamide, nilutamide, enzalutamide, apalutamide, darolutamide or keto-darolutamide, or an AR degrader such as ARV-110, or an AR N-terminal domain binder such as EPI-506, or an antisense oligonucleotide that reduces AR expression such as EZN-4176 or AZD-5312, or an androgen synthesis inhibitor such as abiraterone, particularly abiraterone acetate, seviteronel, galeterone, orteronel or ketoconazole, or a dual AR antagonist and androgen synthesisA inhibitor such as ODM-204, preferably for component B being enzalutamide or darolutamide, include those described in the NCCN Clinical Practice Guidelines in Oncology (NCCN guidelines), in particular in the NCCN Guidelines in Oncology, Version 1.2014.

Further, suitable dose(s), administration regime(s) and administration route(s) for component B may be readily determined by standard techniques known to the skilled person.

The dose(s), administration regime(s) and administration route(s) may have to be adapted according to, inter alia, the indication, the indication stage, the patient age and/or the patient gender, among other factors. Such adaptations can be readily determined by standard techniques known to the skilled person. For both, for the ATR kinase inhibitors, particularly Compound A, and for the antiandrogen the administered dosage of the compound(s) may be modified depending on any superior or unexpected results which may be obtained as routinely determined with this invention.

PSMA-TTC and the antiandrogen can be administered to a patient orally, topically, parenterally, rectally, by inhalation, and by injection. Administration by injection includes intravenous, intramuscular, subcutaneous, and parenterally as well as by infusion techniques. The agents can be administered by any of the conventional routes of administration for these compounds. The preferred route of administration for the ATR kinase inhibitor and the antiandrogen is typically orally, which is the same route of administration used for each agent alone. Any of the antiandrogens described supra can be administered in combination with a compound of general formula (I) or (Ib) described supra, particularly with Compound A, by any of the mentioned routes of administration.

For administering PSMA-TTC, and the antiandrogen by any of the routes of administration herein discussed, PSMA-TTC, can be administered simultaneously with the antiandrogen. This can be performed by administering a single formulation which contains both PSMA-TTC and the antiandrogen. Alternatively, this can be performed by administering PSMA-TTC, and the antiandrogen in independent formulations at the same time to a patient.

Alternatively, PSMA-TTC described supra, can be administered in tandem with the antiandrogen. PSMA-TTC can be administered prior to the antiandrogen. For example, PSMA-TTC, can be administered once every 6 weeks for 6 cycles followed by administration of the antiandrogen described supra. Also, the antiandrogen as described supra can be administered first once or more times per day up to 28 consecutive days followed by administration of the PSMA-TTC. The choice of sequence administration of the PSMA-TTC, relative to the antiandrogen may vary for different agents. Also, the antiandrogen described supra can be administered using any regimen which is conventionally used for these agents.

In another regimen of administration, PSMA-TTC, and the antiandrogen can be administered once or more times per day on the day of administration.

Any of the routes and regimens of administration may be modified depending on any superior or unexpected results which may be obtained as routinely determined with this invention.

### EXPERIMENTAL SECTION

Component A:

In this Experimental Section and in the Figures, the term “compound A” refers to PSMA-TTC, which is described above.

Compound A: PSMA-TTC is BAY 2315497 and is prepared according to Example 9, specifically Examples 9a and 9b of WO 2016/096843. The monoclonal antibody is AB-PG1-XG1-006 as disclosed in WO 03/034903

Component B:

Compound B used in the Examples below is darolutamide (=ODM-201)

Compound B′ used in the Examples below is Enzalutamide.

TABLE 1
Test system

| Cell line/Tumor | | |
|---|---|---|
| model | Tumor entity | Source |
| LNCaP | Prostate carcinoma | ATCC |
| C4-2 | Prostate carcinoma | ATCC/MD Anderson |
| ST1273 | Prostate carcinoma | South Texas Accelerated |
| | | Research Therapeutics |
| | | (START) |
| KUCaP-1 | Prostate carcinoma | Prof. O. Ogava, University |
| | | of Kyoto, Japan |

### Example 1

### Synergistic In Vitro Cytotoxicity

The effects of PSMA-TTC compound A′ and compound B (darolutamide, ODM-201) or compound C (enzalutamide) in PSMA overexpressing prostate cancer cell lines LNCaP and C4-2 were investigated in vitro. LNCaP (hormone sensitive) and C4-2 (hormone insensitive) cells were co-cultured in presence of either enzalutamide or darolutamide/ODM-201 and the effect on cell binding of PSMA-conjugate and cytotoxicity of PSMA-TTC was evaluated.

C4-2 and LNCaP cells were seeded in similar cell medium (RPMI/10% FBS/1% HEPES/1% MEM NEAA/1% NaPyruvate/1% L-glutamine). One day after seeding, cells were exposed to either enzalutamide (1 μM), darolutamide (=ODM-201) (10 μM) or DMSO (0.1% (v/v); reference). Seven, 14 and 21 days after co-culturing, the cells were analyzed for cell surface binding of PSMA antibody-chelator conjugate by flow cytometry. The binding of PSMA conjugate at saturating concentrations increased in both cell lines after treatment with the AR antagonists: maximal binding of PMSA conjugate was increased by 1 μM enzalutamide by 1.9 to 2.8 fold and by 10 μM darolutamide by 1.7-3.6 fold. Similar results were obtained in LNCaP cells. In addition, cells pre-treated in the presence or absence of AR antagonists at the respective concentrations entered cytotoxicity experiments using cell titer Glo (Promega) as readout for cell viability.

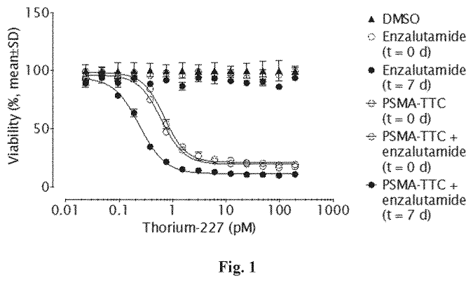

Respective cells pretreated for 0 or 7 days with AR antagonists were seeded on Day −1 at an appropriate cell density (e.g. 1000 cells/well). On day 0, PSMA-TTC at specific activity of 50 MBq/mg was titrated simultaneously in presence or absence of fixed concentrations (1 or 10 μM) of and the AR antagonists. Cell viability was measured after incubation for 5 days in the presence of dose titrations of PSMA-TTC or corresponding controls. Incubation of C4-2 cells in the presence of enzalutamide (1μM) or darolutamide (10 μM) for 7 days followed by dose titration of PSMA-TTC resulted in increased sensitivity to growth inhibition by PSMA-TTC (FIG. 1).

### Example 2

### In Vivo Xenotransplantation of Human Patient Derived Prostate Cancer Models

The anti-tumor activity of combination treatment of Compound A and Compound B (Darolutamide) or Compound A and Compound B′ (=Enzalutamide) was evaluated in the human patient derived prostate cancer models ST1273 and KUCaP-1. To circumvent unspecific uptake of the test compound by organs such as the spleen, all mice were pre-dosed with 200 μg of an irrelevant mouse antibody (IgG2a-kappa murine myeloma monoclonal UPC10 antibody, Sigma-Aldrich, St. Louis, Mo., USA) i.v., 16-24 h prior to treatment with TTCs. PSA levels in blood were determined by ELISA from tumor bearing animals.

Female NMRI nude (RjOrl:NMRI-Foxn1nu/Foxn1nu) mice (23.9-33.7 g, 6-8 weeks, Janvier Labs) supplemented with testosterone (Testosterone MedRod 100 μg/day, PreclinApps, Raisio, Finland) were implanted subcutaneously with 5×5×5 mm ST1273 human PrCa tumor fragments (n=10 mice/group). Mice were subsequently injected intravenously with vehicle (30 mM citrate, 70 mM NaCl, 0.5 mg/mL PABA, 2 mM EDTA, pH 5.5), PSMA-TTC (250 kBq/kg at 014 mg/kg total antibody dose), enzalutamide (30 mg/kg, QD×28, p.o.) or the combination of PSMA-TTC and enzalutamide at the respective doses on day 28 (tumor size 100-200 mm3).

Male CB17-Scid mice (20 g, 5-6 weeks, Janvier Labs) were implanted subcutaneously with 5×5×5 mm KUCaP-1 human PrCa tumor fragments (n=10 mice/group). The mice were subsequently injected intravenously with vehicle (isotonic saline; 0.9% NaCl, Baxter), PSMA-TTC (150 kBq/kg, 0.43 mg/kg total antibody dose), darolutamide (200 mg/kq, QD, p.o.) of the combination of PSMA-TTC and darolutamide on day 23 (average tumor size 150 mm3).

Subcutaneous tumor growth was monitored by measuring tumor volume (0.5×length×width2) using a caliper unless mentioned otherwise. Animal body weight was monitored as an indicator of treatment-related toxicity. Measurement of tumor volume and body weight was performed two to three times per week. Individual animals were sacrificed when showing >20% body weight loss or when tumors reached a maximum size of ˜1000 mm3. At study termination, the animals were sacrificed by cervical dislocation under CO2-anesthesia or equal. T/C (treatment/control) ratios were calculated using final tumor volume on the last day of the control group. In addition, final tumor volumes of combination treatment groups were compared to the respective monotherapy values.

Results:

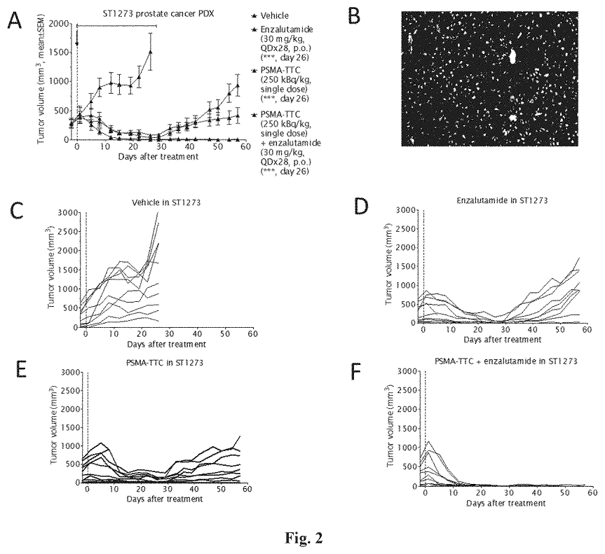

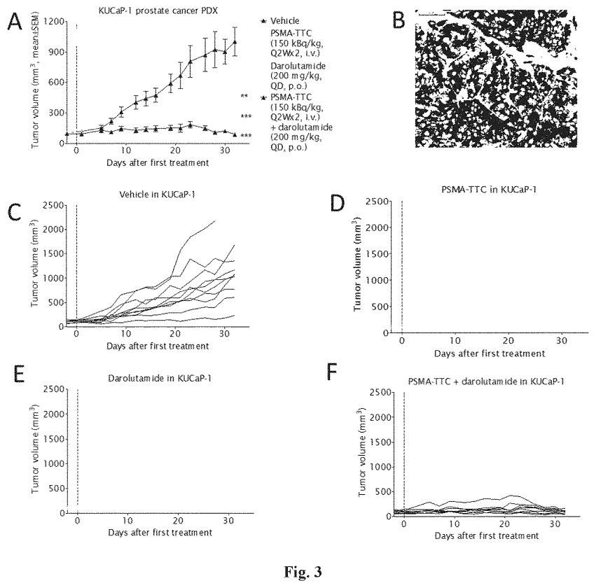

In the ST1273 model treated with a single i.v. injection of PSMA-TTC, complete response was observed in 20% and partial response in 80% of the mice 26 days after dosing, with a T/C ratio of 0.05. With daily enzalutamide treatment, complete and partial response rates of 33% and 67% were observed (T/C ratio 0.02). Combining PSMA-TTC and enzalutamide resulted in tumor reduction in all mice, with complete response observed in 44% and partial response in 56% of the mice 26 days after start of treatment (T/C ratio 0.002) (FIG. 2 and Table 2). No significant adverse effects on body weight were detected compared to vehicle-treated animals in any of the groups. In the KUCaP-1 model, both PSMA-TTC and darolutamide monotherapies were efficacious with T/C ratios of 0.28 and 0.47, respectively. The combination of PSMA-TTC with darolutamide (T/C ratio 0.09) resulted in partial response or stable disease in 56% of the animals up until 32 days after start of treatment (FIG. 3 and Table 2). No significant adverse effects on body weight were detected compared to vehicle-treated animals in any of the groups.

In both models combination treatments of PSMA-TTC (Compound A) with AR antagonists Compound B (=Darolutamide) or Compound B′ (=Enzalutamide) showed improved anti-tumor efficacy with regard to T/C values as well as response rates compared to the respective monotherapies.

TABLE 2
Anti-tumor activity of Compound A, Compound B or Compound B′ in monotherapy as well
as combination of Compound A and Compound B or Compound A and Compound C based on tumor
volumes in the human ST1273 and KuCaP-1 patient derived prostate cancer xenograft model in mice.

| | PSMA | Efficacy^{a} (T/C ratio) | | | Response rate^{a} | | |
|---|---|---|---|---|---|---|---|
| Cell | expr. in tissue | PSMA-TTC | AR ant. | PSMA-TTC + | PSMA-TTC | AR antagonist | PSMA-TTC + |
| line | (IHC score) | monotherapy | monotherapy | AR ant. | monotherapy | monotherapy | AR antagonist |
| ST1273 | 3+ | 0.05*** | 0.02*** | 0.002*** | RR: 100% | RR: 100% | RR: 100% |
| | | | | | (CR: 2/10, | (CR: 3/9, | (CR: 4/9, |
| | | | | | PR: 8/10) | PR: 6/9) | PR: 5/9) |
| KUCaP-1 | 3+ | 0.28*** | 0.47** | 0.09*** | RR: 0% | RR: 0% | RR: 44% |
| | | | | | (PD: 10/10) | (PD: 10/10) | (PR: 4/9, |
| | | | | | | | SD: 1/9, |
| | | | | | | | PD: 4/9) |

^{a}Response rates and T/C ratios were calculated on day 26 for ST1273 and on day 32 for KUCaP-1
Statistical analysis was performed using linear models estimated with generalised least squares that included separate variance parameters for each study group. Mean comparisons between the treatment and control groups were performed using the estimated linear model and corrected for family-wise error rate using Sidak's method.
**p < 0.01;
***p < 0.001.
IHC, immunohistochemistry;
RR, response rate;
T/C, treatment/control;
AR, androgen receptor;
CR, complete response;
PR: partial response,
SD; stable disease,
PD, progressive disease

FIG. 1 shows In vitro cytotoxicity of PSMA-TTC in C4-2 prostate cancer cells with or without co-treatment with enzalutamide (1p M). Enzalutamide treatment was started at the same time as PSMA-TTC (t=0 d) or 7 days before (t=7 d).

FIG. 2 shows antitumor efficacy of PSMA-TTC and enzalutamide in the ST1273 prostate cancer PDX model in mice. Mice were treated with PSMA-TTC (250 kBq/kg, single dose, i.v., at total antibody dose of 0.14 mg/kg), enzalutamide (30 mg/kg, QD×28, p.o.) or their combination.

A. Growth curves of ST1273 tumors, presented as mean tumor volumes. PSMA-TTC treatment day is indicated with green arrows and enzalutamide with pink arrows. Statistical analysis was performed using linear models estimated with generalised least squares that included separate variance parameters for each study group. Mean comparisons between the treatment and control groups were performed using the estimated linear model and corrected for family-wise error rate using Sidak's method.

B. PSMA expression in vehicle-treated ST1273 PDX tumors as determined by IHC.

Tumor growth curves of individual mice treated with (C) vehicle, (D) PSMA-TTC, (E) enzalutamide, and (F) combination of PSMA-TTC and enzalutamide.

FIG. 3 illustrates antitumor efficacy of PSMA-TTC and darolutamide in the KUCaP-1 prostate cancer PDX model in mice. Mice were treated with PSMA-TTC (125 kBq/kg, Q2W×2, i.v., at a total antibody dose of 0.43 mg/kg), darolutamide (200 mg/kg, QD, p.o.) or their combination.

A. Growth curves of KUCaP-1 PDX tumors in mice, presented as mean tumor volumes. PSMA-TTC treatment days are indicated with green arrows and darolutamide with blue arrows. Statistical analysis was performed using linear models estimated with generalised least squares that included separate variance parameters for each study group. Mean comparisons between the treatment and control groups were performed using the estimated linear model and corrected for family-wise error rate using Sidak's method.

B. PSMA expression in vehicle-treated KUCaP-1 PDX tumors as determined by IHC.

Tumor growth curves of individual mice treated with (C) vehicle, (D) PSMA-TTC, (E) darolutamide, and (F) combination of PSMA-TTC and darolutamide.

1. A combination, comprising a component A, wherein component A is PSMA-TTC, and component B, wherein component B is an antiandrogen.

2. The combination according to claim 1, wherein the antiandrogen is selected from AR antagonists, an AR degrader, an AR N-terminal domain binder, an antisense oligonucleotide that reduces AR expression, an androgen synthesis inhibitor, or a dual AR antagonist and androgen synthesis inhibitor.

3. The combination according to claim 1, wherein the antiandrogen is selected from the group consisting of bicalutamide, enzalutamide, apalutamide, abiraterone acetate and darolutamide (ODM-201).

4. The combination according to claim 1, wherein the antiandrogen is selected from the group consisting of enzalutamide and darolutamide (ODM-201).

5. A method for treatment or prophylaxis of a hyper-proliferative disease in a subject, comprising administering to said subject a therapeutically effective amount of a combination according to claim 1.

6. (canceled)

7. The method according to claim 5, wherein the hyper-proliferative disease is selected from the group consisting of prostate cancer and breast cancer.

8. A kit, comprising
a combination according to claim 1, wherein both or either of PSMA-TTC and the antiandrogen are in the form of a pharmaceutical composition which is ready for use to be administered simultaneously, concurrently, separately or sequentially.

9. (canceled)

10. A pharmaceutical composition, comprising a combination according to claim 1, and one or more pharmaceutically acceptable excipients.

11. The pharmaceutical composition according to claim 10, wherein PSMA-TTC and the antiandrogen are present in a joint formulation.

12. The pharmaceutical composition according to claim 10, wherein PSMA-TTC and the antiandrogen are present in separate formulations.

13. The combination according to claim 2, wherein the antiandrogen is cyproterone acetate, bicalutamide, flutamide, nilutamide, enzalutamide, apalutamide, darolutamide, keto-darolutamide, ARV-110, EPI-506, EZN-4176, AZD-5312, abiraterone, seviteronel, galeterone, orteronel or ketoconazole, or ODM-204.

14. The kit according to claim 8, wherein the kit comprises component C, wherein component C is one or more further pharmaceutical agents.

15. The kit according to claim 14, wherein all, both or either of said components A and B and C are in the form of a pharmaceutical composition which is ready for use to be administered simultaneously, concurrently, separately or sequentially.
